(19) United States
(12) Patent Application Publication
(10) Pub. No.: US 2010/0062431 A1
(43) Pub. Date: Mar. 11, 2010

(54) USE OF ADAMTS4 GENE AND PROTEIN POLYMORPHISMS

(75) Inventors: Detlef Kozian, Kelkeim (DE); Matthias Herrmann, Hofheim (DE); Karl-Ernst Siegler, Ludwigshafen (DE); Jean-Francois Deleuze, Combs La Ville (FR); Sylvain Ricard, Paris (FR); Sandrine Mace, Jouy-En-Josas (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/300,200

(22) PCT Filed: May 9, 2007
(86) PCT No.: PCT/EP2007/004095

§ 371 Date: Mar. 10, 2009

(30) Foreign Application Priority Data
May 18, 2006 (DE) 102006023253.4

(51) Int. Cl. C12Q 1/68; C07H 21/04; C12N 9/50

(52) U.S. Cl. 435/6; 536/24.31; 536/23.2; 435/219; 536/24.33

(57) ABSTRACT

The use of the single nucleotide polymorphism (SNP) of the ADAMTS4 gene for the identification of cardiovascular and peripheral vascular disorders or of an increased risk for developing cardiovascular and peripheral vascular disorders in a biological sample taken from an individual to be examined; the use of ADAMTS4 for identifying substances active in preventing and/or treating cardiovascular and peripheral vascular disorders and methods for doing so.

The invention relates to the use of single nucleotide polymorphisms (SNPs) and protein polymorphisms for identifying an increased risk of cardiovascular and peripheral vascular disorders and to primers and nucleic acids suitable for said use. In addition, the invention relates to the use of ADAMTS4 (a disintegrin and metalloprotease with thrombospondin motifs) for finding active substances for preventing and treating cardiovascular and peripheral vascular disorders.

In the western world, cardiovascular and peripheral vascular disorders are among the leading causes of death among both sexes. Cardiovascular disorders comprise all disorders affecting the function of the heart and include especially disorders of the cardiac tissue and the cardiac vessels. A peripheral vascular disorders may be any arterial, venous, arterio-venous or lymphatic disorder of occlusive or functional character outside of the heart and the term comprises, e.g. peripheral vascular disease (PVD). PVD is the build-up of atherosclerotic plaque in the arteries outside of the heart.

Coronary heart disease, in particular coronary artery disease, can be regarded as one of the major causes of cardiovascular disorders. Angina, also called angina pectoris, is temporary chest pain or a sensation of pressure that occurs when the heart muscle is not supplied with enough oxygen. When the coronary arteries are narrowed or blocked so that blood flow to the heart muscle cannot increase to meet the increased demand for oxygen, ischemia may occur as a result thereof, causing said pain (i.e. said angina pectoris). Normally, angina pectoris results from coronary artery disease but may also be caused by other coronary heart diseases. Not every ischemia of the heart muscle causes the pain or sensations of pressure connected with angina pectoris. Ischemia of the heart muscle of this kind, i.e. without angina pectoris, is referred to as silent ischemia. The danger of silent ischemia lies in the fact that the damage to the heart muscle is not noticed by the individual affected. Therefore, the patient or the physician in charge are often unable to recognize possible damage to the heart tissue, until said damage ultimately results in a myocardial infarction. For this reason, there is a great demand for diagnostic methods and means for recognizing vascular and cardiac degenerations, which enable a diagnosis, and thus a therapeutic intervention, as early as possible.

Hypertension (arterial hypertension) is an elevation of the systolic and/or diastolic blood pressure (systolic blood pressure more/equal to 140 mmHg and/or diastolic blood pressure more/equal to 90 mm Hg) and can be either of primary or secondary nature. The etiology of primary hypertension is so far unknown; certainly it is not caused by a single factor, heredity being a predisposing factor and acting, e.g. together with environmental factors and nutrition. Secondary hypertension is associated with certain types of renal disease or other disorders, e.g. certain hormonal disorders. Untreated hypertension severely increases the risk of experiencing a left ventricular heart failure, heart infarction, cerebral hemorrhage and other possibly fatal diseases, at an early age. Heart infarction, also called myocardial infarction, is the irreversible necrosis of heart muscle tissue which is caused by prolonged ischemia of the tissue.

Stroke is a sudden disruption of the brain's blood supply (ischemia of the brain), of which normally a definite brain area is affected and which results in neurological disorders, the character of which depends on the brain area affected. A transitoric ischemic attack (TIA) is a temporary neurological disorder having similar causes as a stroke but the symptoms of which are normally completely reversible. TIAs take several minutes to hours (per definition at maximum 24 hours) and hint to the possible onset of a stroke (in 51% within the consecutive year and in 21% of the cases within the following months). Prolonged reversible ischemic neurological deficit (PRIND) is a disturbance of the brain's blood supply proceeding delayed, the symptoms of which occur normally within 24 to 28 hours. Like at a TIA, the probability of suffering from a stroke are increased after experiencing a PRIND.

Due to the high socio-economic and personal burdens associated with cardiovascular and peripheral vascular diseases, there is a great need for early diagnosis and treatment of said disorders.

It is thus the object of the present invention to provide improved methods for the diagnosis and treatment of cardiovascular and peripheral vascular disorders.

According to the invention, this object is achieved by using one or more of the single nucleotide polymorphisms (SNPs) or in the ADAMTS4 gene or polymorphisms of the ADAMTS4 protein for the identification of cardiovascular and peripheral vascular disorders or of an increased risk for developing cardiovascular and peripheral vascular disorders in a biological sample taken from an individual to be examined.

This can e.g. be achieved by analyzing an ADAMTS4 nucleic acid or protein for the presence of the single nucleotide polymorphisms at one or more of the positions 635, 820, 835, 2564 and 10570 of the ADAMTS4 nucleic acid sequence and/or protein polymorphisms at one or more of the positions 77 and 720 of the ADAMTS4 protein or analyzing the amount of protein or mRNA of ADAMTS4 present in the sample taken.

The type of the nucleotide or amino acid at any relevant position within the ADAMTS4 nucleic acid or protein can be determined here on the basis of common methods. By knowing the type of the nucleotide or amino acid at certain positions, the skilled worker can readily determine the risk group to which the individual belongs from whom the sample was derived.

ADAMTS4 belongs to a family of extracellular proteases comprising at least 14 members and is also referred to as Aggrecanase-1. The gene for ADAMTS4 is located on chromosome 1q21-q23 and encodes an 837 amino acid protein consisting of a signal sequence, a pro-peptide domain, a disintegrin-like domain and a c-terminal domain with thrombospondine type I motif. Substrates of ADAMTS4 comprise the extracellular matrix proteins Aggrecan, Brevican and Versican (Int J. Biochem. Cell Biol. 33(1): 33-44, 2001; Curr Opinion in Pharmacol 2: 322-329, 2002). So far, the role and function of the aggrecanase ADAMTS4 has been studied in the context of joint diseases, since it acts as protease of Aggrecan, the loss and degradation of which in the articulate cartilage is directly connected with pathophysiological consequences. The expression of ADAMTS4 may be regulated on the transcriptional and/or post-transcriptional level and it may be differentially expressed upon stimuli. Activators of ADAMTS4 function comprise TGFβ, IL-1, TNFalpha, fragments of fibronectine (for an overview, see Curr Opinion in Pharmacol 2: 322-329, 2002 and the references cited therein) and glycosylphosphatidylinositol-anchored membrane type 4-metalloproteinase (MT4-MMP, MMP-17; JBC 2004, Vol. 279, p. 10042-10051). These may either act by modulating ADAMTS4 expression or by activation of constitutively produced ADAMTS4 protein (via inhibition of expression or function of an ADAMTS4 inhibitor or activation of expression or function of an ADAMTS4 activator), as shown for IL-1, Arthritis & Rheumatism, 2003, Vol. 48, p 119-133. IL-1 obviously exerts its modulatory effect on ADAMTS4 by influencing the c-terminal processing of ADAMTS4, which has an effect on the specificity and activity of the protease activity of ADAMTS4 (JBC 2004, Vol. 279, p. 10190-10119). Aggrecanase activity of ADAMTS4 is positively affected by C-terminal processing by MT4-MMP and binding of the activated form of ADAMTS4 to chondroitin sulfate or heparan sulfate residues on Syndecan-1 (JBC 2004, Vol. 279, p. 10042-10051). The gene for ADAMTS4 has been isolated originally from a human brain cDNA library and was originally designated KIAA0688 (DNA Res. 5: 169-176, 1998).

The sequence of the ADAMTS4 gene is known in the art. A part of the genomic ADAMTS4 sequence can be retrieved at the NCBI Nucleotide Database under the number NM_{—}005099.2 (SEQ ID No.1). The complete coding nucleic acid sequence of said gene can be retrieved under the number AY044847.1 (SEQ ID No.2). The derived protein sequence (SEQ ID No.3) is accessible under the number NM_{—}005099.3 at the NCBI Nucleotide Database and the mRNA sequence (SEQ ID No.16) likewise under the number NM_{—}005099.3. NCBI is the National Center for Biotechnology Information (postal address: National Center for Biotechnology Information, National Library of Medicine, Building 38A, Bethesda, Md. 20894, USA; web address: www.ncbi.nhm.nih.gov).

The present group of inventions related to one another is based on studies of the ADAMTS4 gene at the chromosomal level in a clinical cohort of patients, carried out by the inventors to estimate the influence of variations in the ADAMTS4 gene and/or protein on the clinical and phenotypic picture of a carrier of such variants.

Single nucleotide polymorphisms (SNPs) are variants of a particular nucleotide sequence containing substitutions at individual positions and are well known to the skilled worker. The term protein polymorphism as used herein comprises any change of the protein primary (i.e. amino acid sequence), secondary (i.e. protein folding) and/or tertiary structure (i.e. the assembly of a protein from different polypeptide subunits) and preferably comprises changes caused by one or more SNPs of the gene encoding a protein; examples are, e.g. amino acid exchanges, amino acid deletions or truncations of the protein.

Different single nucleotide polymorphisms (SNPs) of the ADAMTS4 gene are known in the prior art and publicly accessible at the NCBI database, e.g. at http://www.ncbi.nlm.nih.qov/SNP/snp ref.cqi?locusId=9507.

However, the gene polymorphisms in the ADAMTS4 gene, on which the invention is based, and the association of said polymorphisms ADAMTS4 with a predisposition for developing cardiovascular and peripheral vascular disorders have not been known, so far.

Experiments of the inventors have demonstrated for the first time that certain variations in the ADAMTS4 gene occur with statistically significant frequency in humans suffering from cardiovascular and peripheral vascular disorders. To assess a possible relation of SNPs within or variants of the ADAMTS4 gene or protein with the onset and the development of diseases, the genetic variants of ADAMTS4 have been analyzed in detail and genotype-phenotype association analyses have been performed in a well-defined patient cohort. The result is the surprising finding that ADAMTS4 polymorphisms correlate with a predisposition for cardiovascular and peripheral vascular disorders. The correlation of polymorphisms of the ADAMTS4 gene with a predisposition for this type of disease has never been described before and, in view of the known function of ADAMTS4 in relation to of joint diseases, is to be regarded as completely surprising.

The different aspects of present invention are applicable for all animals or human beings. A preferred embodiment concerns the application for mammals and/or humans. Accordingly, the term ADAMTS4 (with respect to nucleic acid, protein, polymorphisms etc.) refers to ADAMTS4 from any animal species or homo sapiens. Preferred embodiments encompass ADAMTS4 from mammals and/or homo sapiens (hs) ADAMTS4.

Thus, another aspect of present invention concerns the use of an ADAMTS4 protein or nucleic acid or of a functional fragment thereof for the identification of cardiovascular and peripheral vascular disorders or of an increased risk for developing cardiovascular and peripheral vascular disorders in a biological sample taken from an individual to be examined.

In the following, the most frequently occurring nucleotide or amino acid occurring at a given position within the ADAMTS4 gene and/or nucleic acid or protein is referred to as the most frequent variant or the “wild type”. If, therefore, reference is made in the present application of the substitution or mutation of an nucleobase or an amino acid at the site in question within the ADAMTS4 gene and/or nucleic acid or protein, or of a less frequent variant of the ADAMTS4 gene and/or nucleic acid or protein, then this relates to the presence of a nucleobase or amino acid other than the wild type or most frequent variant at the given position within the ADAMTS4 gene, nucleic acid or protein.

ADAMTS4-G635G describes the group of persons who have a Guanosine (G) at position 635 with respect to the reference sequence NM_{—}005099.2 (SEQ ID No.1) on both alleles of the ADAMTS4 gene. This polymorphism leads to an ADAMTS4 protein having the amino acid Alanine (Ala or A) at position 77 (Ala77) with respect to the reference sequence SEQ ID 3 (protein sequence derived from NM_{—}005099.3). Said persons are homozygous with respect to said ADAMTS4 variant. As can be gained from table 5, the nucleotide G is the most frequent variant at position 635 of the ADAMTS4 gene and the amino acid Alanine the most frequent variant at position 77 of the ADAMTS4 protein.

ADAMTS4-G635A describes the group of persons having an Guanosine (G) at position 635 on one allele of the ADAMTS4 gene with respect to the reference sequence NM_{—}005099.2 (SEQ ID No.1), leading to the amino acid Alanine (Ala or A) at position 77 with respect to the corresponding protein. This group of persons has an Adenosine (A) at position 635 of the other allele of the ADAMTS4 gene with respect to the reference sequence NM_{—}005099.2 (SEQ ID No.1) leading to the amino acid Threonine (Thr or T) at position 77 (Thr77) of the ADAMTS4 protein. These persons are heterozygous with respect to said ADAMTS4 variant.

ADAMTS4-A635A describes the group of persons who have an Adenosine (A) at position 635 with respect to the reference sequence NM_{—}005099.2 (SEQ ID No.1) on both alleles of the ADAMTS4 gene, leading to the amino acid Threonine (Thr or T) at position 77 (Thr77) of the ADAMTS4 protein. Said persons are homozygous with respect to said ADAMTS4 variant.

ADAMTS4-G820G describes the group of persons who have a Guanosine (G) at position 820 with respect to the reference sequence NM_{—}005099.2 (SEQ ID No.1) on both alleles of the ADAMTS4 gene. This polymorphism leads to an ADAMTS4 protein having the amino acid Threonine (Thr or T) at position 131 (Thr131) with respect to the reference sequence SEQ ID 3 (protein sequence derived from NM_{—}005099.3). Said persons are homozygous with respect to said ADAMTS4 variant. As can be gained from table 3, the nucleotide T is the most frequent variant at position 820 of the ADAMTS4 gene.

ADAMTS4-G820A describes the group of persons having an Guanosine (G) at position 820 on one allele of the ADAMTS4 gene with respect to the reference sequence NM_{—}005099.2 (SEQ ID No.1), leading to the amino acid Threonine (Thr or T) at position 131 (Thr181) with respect to the corresponding protein. This group of persons has an Adenosine (A) at position 820 of the other allele of the ADAMTS4 gene with respect to the reference sequence NM_{—}005099.2 (SEQ ID No.1). The amino acid at position 131 of the ADAMTS4 protein stays the same. These persons are heterozygous with respect to said ADAMTS4 variant.

ADAMTS4-A820A describes the group of persons who have an Adenosine (A) at position 820 with respect to the reference sequence NM_{—}005099.2 (SEQ ID No.1) on both alleles of the ADAMTS4 gene. Said persons are homozygous with respect to said ADAMTS4 variant.

ADAMTS4-C835C describes the group of persons who have a Cytidine (C) at position 835 with respect to the reference sequence NM_{—}005099.2 (SEQ ID No.1) on both alleles of the ADAMTS4 gene. This polymorphism leads to an ADAMTS4 protein having the amino acid Proline (Pro or P) at position 136 (Pro136) with respect to the reference sequence SEQ ID 3 (protein sequence derived from NM_{—}005099.3). Said persons are homozygous with respect to said ADAMTS4 variant. As can be gained from table 2, the nucleotide C is the most frequent variant at position 835 of the ADAMTS4 gene.

ADAMTS4-C835T describes the group of persons having a Cytidine (C) at position 835 on one allele of the ADAMTS4 gene with respect to the reference sequence NM_{—}005099.2 (SEQ ID No.1), leading to the amino acid Proline (Pro or P) at position 136 (Pro136) with respect to the corresponding protein. This group of persons has a Thymidine (T) at position 835 of the other allele of the ADAMTS4 gene with respect to the reference sequence NM_{—}005099.2 (SEQ ID No.1). The amino acid at position 136 of the ADAMTS4 protein stays the same. These persons are heterozygous with respect to said ADAMTS4 variant.

ADAMTS4-T835T describes the group of persons who have a Thymidine (T) at position 835 with respect to the reference sequence NM_{—}005099.2 (SEQ ID No.1) on both alleles of the ADAMTS4 gene. Said persons are homozygous with respect to said ADAMTS4 variant.

ADAMTS4-C2564C describes the group of persons who have a Cytidine (C) at position 2564 with respect to the reference sequence NM_{—}005099.2 (SEQ ID No.1) on both alleles of the ADAMTS4 gene. This polymorphism leads to an ADAMTS4 protein having the amino acid Proline (Pro or P) at position 720 (Pro720) with respect to the reference sequence SEQ ID 3 (protein sequence derived from NM_{—}005099.3). Said persons are homozygous with respect to said ADAMTS4 variant. As can be gained from table 1, the nucleotide C is the most frequent variant at position 2564 of the ADAMTS4 gene and respectively the amino acid Proline at position 720 of the ADAMTS4 protein.

ADAMTS4-C2564G describes the group of persons having a Cytidine (C) at position 2564 on one allele of the ADAMTS4 gene with respect to the reference sequence NM_{—}005099.2 (SEQ ID No.1), leading to the amino acid Proline (Pro or P) at position 720 (Pro720) with respect to the corresponding protein. This group of persons has a Guanosine (G) at position 2564 of the other allele of the ADAMTS4 gene with respect to the reference sequence NM_{—}005099.2 (SEQ ID No.1), leading to the amino acid Alanine (Ala or A) at position 720 (Ala 720) of the corresponding protein. These persons are heterozygous with respect to said ADAMTS4 variant.

ADAMTS4-G2564G describes the group of persons who have a Guanosine (G) at position 2564 with respect to the reference sequence NM_{—}005099.2 (SEQ ID No.1) on both alleles of the ADAMTS4 gene. This polymorphism leads to an ADAMTS4 protein having the amino acid Alanine (Ala or A) at position 720 with respect to the reference sequence SEQ ID 3 (protein sequence derived from NM_{—}005099.3). Said persons are homozygous with respect to said ADAMTS4 variant.

ADAMTS4-A10570A describes the group of persons who have an Adenosine (A) at position 10570 with respect to the reference sequence AY044847.1 (SEQ ID No.2) on both alleles of the ADAMTS4 gene. Said persons are homozygous with respect to said ADAMTS4 variant. Since position 10570 is located within the 3′-UTR (untranslated region), it does not have any influence on the amino acid sequence of the ADAMTS4 protein. As can be gained from table 4, the nucleotide A is the most frequent variant at position 10570 of the ADAMTS4 gene.

ADAMTS4-A10570G describes the group of persons having Guanosine (G) at position 10570 on one allele of the ADAMTS4 gene with respect to the reference sequence AY044847.1 (SEQ ID No.2), and an Adenosine (A) at position 10570 of the other allele of the ADAMTS4 gene with respect to the reference sequence AY044847.1 (SEQ ID No.2). These persons are heterozygous with respect to said ADAMTS4 variant.

ADAMTS4-G10570G describes the group of persons who have a Guanosine (G) at position 10570 with respect to the reference sequence AY044847.1 (SEQ ID No.2) on both alleles of the ADAMTS4 gene. Said persons are homozygous with respect to said ADAMTS4 variant.

Genetic variations in the ADAMTS4 gene may be detected, for example:
a) By direct detection of genetic variations at the chromosomal DNA level by way of molecular-biological analysis of the ADAMTS4 gene which may contain said genetic variations, here in particular the regions around positions 635, 820, 835, 2564 at the reference sequence NM_{—}005099.2 or around position 10570 of the reference sequence AY044847.1 of the ADAMTS4 gene.
b) Via detection by measuring ADAMTS4 mRNA expression.
c) By detection of protein polymorphisms within the ADAMTS4 protein, here in particular at positions 77 and/or 720 of the ADAMTS4 polypeptide chain.
d) By indirect detection by way of determining the amounts and/or activity of ADAMTS4 protein present in cells, tissues or body fluids by means of protein-chemical methods.

Genetic variations or polymorphisms at the nucleic acid level (here chromosomal DNA) in the ADAMTS4 gene at one of the above positions within one of the above reference sequences may be detected, for example, by

1) Methods based on the sequencing of the nucleic acid sequence of said region of the ADAMTS4 gene (e.g. pyrosequencing, sequencing using radio labeled or fluorescent dye-labeled nucleotides or via mass spectrometric analysis of said nucleic acid sequence);

2) Methods based on hybridization of nucleic acid sequences of said region of the ADAMTS4 gene (e.g. by means of “DNA micro arrays”);

3) Methods based on the analysis of amplification products of the nucleic acid sequence of said region of the ADAMTS4 gene (e.g. TaqMan analyses).

Genetic variations or polymorphisms at the nucleic acid level (here chromosomal DNA) in the ADAMTS4 gene at one or more of the above positions with respect to one of the above reference sequences may also be detected, for example, on the basis of measuring expressed ADAMTS4 mRNA via

1) Methods based on the hybridization of nucleic acid sequences of the ADAMTS4 gene (e.g. by means of “DNA micro arrays”, Northern blot analyses);

2) Methods based on the analysis of amplification products of the nucleic acid sequence of the ADAMTS4 gene (e.g. “TaqMan” analyses, differential RNA display, representational difference analysis).

In addition, genetic variations or polymorphisms at one or more of the above positions within one of the above reference sequences may be detected via analyzing the amount and/or activity of the ADAMTS4 protein. The amount and/or activity of the ADAMTS4 protein may be detected, for example, on the basis of

1) Methods based on quantitative detection of the amount of the ADAMTS4 protein (e.g. Western blot analyses, ELISA test)

2) Methods based on functional detection of the activity of the ADAMTS4 protein via in vitro test systems, for example in human cells, animal cells, bacteria and/or yeast cells.

The protein polymorphisms within the ADAMTS4 protein at one or both of the positions 77 or 720 with respect to the protein sequence according to the reference sequence NM_{—}005099.3 (SEQ ID No.3) can e.g. be detected by means of specific antibodies being able to discriminate between e.g. an Alanine or a Threonine at position 77 or between a Proline or an Alanine at position 720 within the ADAMTS4 protein.

The detection of the genetic variations or polymorphisms in the ADAMTS4 gene at one of the above positions may be used, for example, as (a) genetic marker for evaluating the risk of cardiovascular and peripheral vascular disorders, e.g.: peripheral vascular disease, high blood pressure, stroke/PRIND/TIA, instable angina, premature myocardial infarction, myocardial infarction and/or coronary heart diseases (b) marker for preventative treatment of cardiovascular and peripheral vascular disorders, e.g.: peripheral vascular diseases, high blood pressure, stroke/PRIND/TIA, instable angina, premature myocardial infarction, myocardial infarction and/or coronary heart diseases in carriers of the corresponding genetic variants, (c) marker for adapting the dosage to be administered of a pharmaceutically active substance for cardiovascular and peripheral vascular disorders, e.g.: peripheral vascular diseases, high blood pressure, stroke/PRIND/TIA, instable angina, premature myocardial infarction, myocardial infarction and/or coronary heart diseases, (d) marker for determining the high throughput screening strategy for identifying a pharmaceutically active substance for cardiovascular and peripheral vascular disorders, e.g.: peripheral vascular diseases, high blood pressure, stroke/PRIND/TIA, instable angina, premature myocardial infarction, myocardial infarction and/or coronary heart diseases (e) marker for identifying the relevant individuals or patients for clinical studies in order to test the compatibility, safety and efficacy of a pharmaceutical substance for cardiovascular and peripheral vascular disorders, e.g.: peripheral vascular diseases, high blood pressure, stroke/PRIND/TIA, instable angina, premature myocardial infarction, myocardial infarction and/or coronary heart diseases, and (f) basis for developing test systems for analyzing the genetic variation in the ADAMTS4 gene at the DNA, RNA or protein level.

The above-mentioned analyses of the inventors showed for the first time the correlation between ADAMTS4 polymorphisms and a predisposition for developing cardiovascular and peripheral vascular disorders.

Thus, another aspect of present invention concerns the use of an ADAMTS4 protein or nucleic acid or of a fragment thereof for the identification of cardiovascular and peripheral vascular disorders or of an increased risk for developing cardiovascular and peripheral vascular disorders in a biological sample taken from an individual to be examined.

Yet another aspect of present invention concerns a method for identifying cardiovascular and peripheral vascular disorders or an increased risk for developing cardiovascular and peripheral vascular disorders in an individual, which comprises examining a sample taken from an individual for the type of nucleotide, which is present on one or more of the positions 635, 820, 835, 2564 and 10570 on one or both alleles of the ADAMTS4 gene, the type of nucleotide present at said one or more positions being indicative of the risk of said individual to suffer from or develop cardiovascular and peripheral vascular disorders.

Present invention thus also relates to a method for identifying cardiovascular and peripheral vascular disorders or an increased risk for developing cardiovascular and peripheral vascular disorders in an individual, which comprises examining a sample taken from an individual for the type of amino acid present at one or both of the positions 77 and 720 of the polypeptide chain of ADAMTS4 protein, the type of amino acid present at said one or more positions being indicative of the risk of said individual to suffer from or develop cardiovascular and peripheral vascular disorders.

According to one embodiment, the invention concerns a method for identifying cardiovascular and peripheral vascular disorders or an increased risk for developing cardiovascular and peripheral vascular disorders in an individual, which comprises examining a sample taken from the individual, as to whether the amount of ADAMTS4 mRNA and/or protein present in said sample is different from that of one or more reference samples.

A reference sample can e.g. be a sample taken from one or more individuals having one or more of the following genomic and/or protein variants:
a. A nucleotide other than Adenosine, and preferably a Guanosine, at position 635 of the genomic ADAMTS4 sequence on one or both alleles of the ADAMTS4 gene;
b. A nucleotide other than Adenosine, and preferably a Guanosine, at position 820 of the genomic ADAMTS4 sequence on one or both alleles of the ADAMTS4 gene;
c. A nucleotide other than Thymidine, and preferably a Cytidine, at position 835 of the genomic ADAMTS4 sequence on both alleles of the ADAMTS4 gene;
d. A nucleotide other than Guanosine, and preferably a Cytidine, at position 2564 of the genomic ADAMTS4 sequence on one or both alleles of the ADAMTS4 gene;
e. A nucleotide other than Guanosine, and preferably an Adenosine, at position 10570 of the genomic ADAMTS4 sequence on one or both alleles of the ADAMTS4 gene;
f. An amino acid other than Alanine, and preferably Threonine, at position 77 of the polypeptide chain of the ADAMTS4 Protein;
g. An amino acid other than Proline, and preferably an Alanine at position 720 of the polypeptide chain of the ADAMTS4 protein
   the presence of a different amount indicating an increased risk of said individual to suffer from or to develop cardiovascular and peripheral vascular disorders.

The change in the amount of ADAMTS4, i.e. the change in ADAMTS4 levels, may be caused here by influencing all levels of expression (transcription, translation, splicing), post-translational modification, transport of the protein or proprotein, or influence on protein stability as well as by influences due to signal transduction pathways acting on ADAMTS4 expression.

Yet another aspect of present invention concerns a method of determining the risk of suffering from cardiovascular and peripheral vascular disorders comprising analysing an isolated sample of an individual for the presence of one or more of the before-mentioned SNPs or protein polymorphisms and calculating the estimated risk on basis of the age of the patient and the type of nucleotide or amino acid present at one or more of the positions 635, 820, 835, 2564 and 10570 in the ADAMTS4 gene or at position 77 or 720 of the ADAMTS4 protein. A basis for the risk determination are the results according to tables 1 to 10, FIGS. 8 to 12 and can also be derived from additional data derived by analyses as described in the examples.

The cardiovascular or peripheral vascular disorder in any of the different embodiments and aspects of present invention can e.g. be peripheral vascular disease, high blood pressure, stroke/PRIND/TIA, instable angina, myocardial infarction, early myocardial infarction and any pathological state that necessitates the undergoing of a coronary angioplasty.

The nucleotide positions indicated herein refer to the position of the nucleotides in the reference sequence NM_{—}005099.2/SEQ ID No.1, NM_{—}005099.3/SEQ ID No.16 or AY044847.1/SEQ ID No.2. With respect to the ADAMTS4 amino acid sequence, the amino acid positions refer to or to the the reference sequence NM_{—}005099.3/SEQ ID No.3 of FIG. 3. The positions start from 1 for the first amino acid or nucleotide of the reference sequence unless, for nucleotide sequences, the number of the nucleotide is preceded by a +or a −, in which case the nucleotide position is given with respect to the site of translation.

Standard abbreviations will be used herein below synonymously for nucleotides and amino acids (i.e. three- or one-letter code).

A nucleic acid can be any oligo- or polynucleotide, the term “oligonucleotide” concerning nucleic acids of 2 to 25 nucleotides and the term “polynucleotide” referring to nucleic acids having 26 and more nucleotides.

In the present application, the terms protein sequence, amino acid sequence and polypeptide sequence can be used synonymously.

So far, no data connecting clinical effects with ADAMTS4 variants in humans have been disclosed. Surprisingly, the studies by the inventors have been able to closely connect the presence of different ADAMTS4 variants, in particular the G635A, A635A, G820A, A820A, C835T, C2564G, G2564G, A10570G and G10570G variants of the ADAMTS4 gene with a predisposition for cardiovascular and peripheral vascular disorders.

The detection of genetic polymorphisms of the ADAMTS4 gene, in particular of one or more of the G635A, A635A, G820A, A820A, C835T, C2564G, G2564G, A10570G and G10570G variants and, particularly preferably, of one or more of the A635A, A820A, C835T, G2564G and G10570G variants may serve, for example, as genetic marker for preventive treatments and preventive measures (medication, lifestyle), (a) in order to delay or even to prevent the onset of cardiovascular and peripheral vascular disorders, such as peripheral vascular disease, high blood pressure, stroke/PRIND/TIA (PRIND stands for prolonged ischemic attack; TIA stands for transitoric ischemic attack), instable angina, myocardial infarction, early myocardial infarction and any pathological state that necessitates the undergoing of a coronary angioplasty, or to alleviate or stop the severity of the later course and the pathological sequelae, or (b) as genetic marker for adjusting a pharmaceutical dosage or (c) as genetic marker for designing a screening for pharmaceuticals or (d) as genetic marker for identifying and, where appropriate, selecting patients in particular treatments or medical studies.

The methods of the invention enable a predisposition for cardiovascular and peripheral vascular disorders to be identified early, thereby making possible the early use of preventive or curative treatment measures, before classical symptoms such as sensations of pain as a result of tissue damage occur: identification of the polymorphism of the invention or of a changed steady state level of ADAMTS4 by the skilled worker in charge, gives a clear indication for the treating or examining physician to screen for an already persisting damage to vessels or heart tissue, or to administer preventive pharmaceuticals, or to suggest a change in lifestyle even before corresponding damage or pain occurs.

In addition, the novel finding of a connection between said variants and the predisposition for cardiovascular and peripheral vascular disorders allows the use of more effective treatments by hinting at a change in the dosage of particular pharmaceuticals or at the necessity of changing the treatment of patients without said mutation in the ADAMTS4 gene.

Accordingly, the present invention also relates to the use of
a) one or more single nucleotide polymorphisms (SNPs) in the ADAMTS4 gene,
b) one or more protein polymorphisms in the ADAMTS4 protein and/or
c) an ADAMTS4 protein or nucleic acid or of a functional fragment thereof for adapting the dosage of a pharmaceutical for the prevention and/or treatment of cardiovascular and peripheral vascular disorders.

Moreover, present invention relates to a method for adapting the dosage of a pharmaceutical for the prevention and/or treatment of cardiovascular and peripheral vascular disorders in an individual, which method comprises examining a taken sample of the individual for
a) the type of the nucleotide, which is present at one or more of the positions 635, 820, 835, 2564 and 10570 on either or both alleles of the ADAMTS4 gene and/or
b) the type of amino acid present at one or both positions 77 and 720 in the ADAMTS4 protein,
   said dosage being adapted dependent of the type of nucleotide or amino acid present at one or more of said positions.

One embodiment comprises examining the sample taken from the individual as to whether either or both alleles of the ADAMTS4 gene have one or more of the following SNPs:
a) An Adenosine at position 635 of the genomic ADAMTS4 sequence
b) An Adenosine at position 820 of the genomic ADAMTS4 sequence
c) A Thymidine at position 835 of the genomic ADAMTS4 sequence
d) A Guanosine at position 2564 of the genomic ADAMTS4 sequence
e) A Guanosine at position 10570 of the genomic ADAMTS4 sequence
   the dosage of the pharmaceutical being decreased or increased in the presence of one or more of these polymorphisms.

Another embodiment comprises examining the sample taken from the individual as to whether either or both alleles of the ADAMTS4 gene have a nucleotide other than those listed above at one or more of the above-listed positions, the dosage of the pharmaceutical being decreased or increased in the presence of another nucleotide. The other nucleotide is preferably
a) a Guanosine at position 635 and/or 820 of the genomic ADAMTS4 sequence,
b) a Cytidine at position 835 and/or 2564 of the genomic ADAMTS4 sequence or
c) an Adenosine at position 10570 of the genomic ADAMTS4 sequence.

According to another embodiment of present invention, the method for adapting the dosage of a pharmaceutical for the treatment and/or prevention of cardiovascular and peripheral vascular disorders of an individual comprises examining a sample taken from the individual, as to whether the amount of ADAMTS4 mRNA and/or protein present in said sample is different from that of one or more reference samples. A reference sample can e.g. be a sample taken from an individual having one or more of the following genomic and/or protein variants:
a. A nucleotide other than Adenosine, and preferably a Guanosine, at position 635 of the genomic ADAMTS4 sequence on one or both alleles of the ADAMTS4 gene;
b. A nucleotide other than Adenosine, and preferably a Guanosine, at position 820 of the genomic ADAMTS4 sequence on one or both alleles of the ADAMTS4 gene;
c. A nucleotide other than Thymidine, and preferably a Cytidine, at position 835 of the genomic ADAMTS4 sequence on both alleles of the ADAMTS4 gene;
d. A nucleotide other than Guanosine, and preferably a Cytidine, at position 2564 of the genomic ADAMTS4 sequence on one or both alleles of the ADAMTS4 gene;
e. A nucleotide other than Guanosine, and preferably a Adenosine, at position 10570 of the genomic ADAMTS4 sequence on one or both alleles of the ADAMTS4 gene;
f. An amino acid other than Threonine, and preferably an Alanine, at position 77 of the polypeptide chain of the ADAMTS4 protein;
g. An amino acid other than Alanine, and preferably a Proline, at position 720 of the polypeptide chain of the ADAMTS4 protein;
said dosage being adapted depending on whether the amount of protein and/or mRNA in the taken sample of the individual is different from that of the reference sample or reference samples from one or more individuals having one or more of the variants as listed above under a to g.

The presence of an ADAMTS4 gene variant, in particular that of the ADAMTS4 G635A, A635A, G820A, A820A, C835T, C2564G, G2564G, A10570G and/or G10570G variant, has indicator function. The prior art knows a multiplicity of pharmaceuticals for treating or preventing cardiovascular and peripheral vascular disorders. Since not all pharmaceuticals have the same effect on all patients with the same disease, patients which are treated with cardiovascular pharmaceuticals for the first time normally have to be “adjusted” to the latter, i.e. the treating physician de facto has to test on the individual patient as to which dosage of which pharmaceutical has the desired effect with, at the same time, side effects as small as possible. The disadvantage here is the fact that it is not known beforehand, whether the symptoms in the patient are alleviated or stopped by the pharmaceutical administered (at the given dosage). It is also not possible beforehand to assess accurately, whether said patient will suffer from an undesired side effect.

In this context, identifying the patients as patients having a certain ADAMTS4 gene variant or having an amount of ADAMTS4 nucleic acid or protein associated with a certain probability of suffering from cardiovascular or peripheral vascular disorders prior to the treatment, may improve the predictability of the success of treatment with a particular pharmaceutical: the connection of particular variants of the ADAMTS4 gene with the occurrence of cardiovascular and peripheral vascular disorders suggests that ADAMTS4 variations of this kind concur with physiological changes in the individual which ultimately have the effect that said individuals have a higher or lower probability of suffering from a cardiovascular and peripheral vascular disorders than other individuals. The different efficacy of each pharmaceutical in different individuals must be seen against such a background of different physiological provision of the individual patients. Assigning an individual to such a group of patients with a particular physiological background would allow particular pharmaceuticals which have been proven in clinical studies to be particularly active here to be preferably used and pharmaceuticals which are less active or more likely linked to undesired side effects in this group of patients, compared to patients without said variant, not to be used from the outset.

Normally, a classification of this kind of individual patients prior to a treatment is not possible. Only the knowledge of the connection between the polymorphism on which the invention is based with the occurrence of cardiovascular disease makes this possible. Thus, pharmaceuticals which have shown good success in the treatment of patient groups having the same gene variant in clinical studies may preferably be used on patients having a variation in the ADAMTS4 gene, whereas pharmaceuticals which are less effective in said patient group or which have a higher probability of undesired side effects than in patient groups having a different gene variant would not be used from the outset. This would reduce the risk for the patient who is “adjusted” to a pharmaceutical and increase the probability of a successful treatment.

Accordingly, a further aspect of present invention relates to the use of
a) one or more single nucleotide polymorphisms (SNP) in the ADAMTS4 gene,
b) one or more polymorphisms in the ADAMTS4 protein and/or
c) an ADAMTS4 protein or nucleic acid or of a fragment thereof
   for identifying individuals responding to a pharmaceutical for the treatment and/or prevention of cardiovascular and peripheral vascular disorders.

Such identification may be carried out, for example, by examining a sample taken from an individual as to whether
a. Either or both alleles of the ADAMTS4 gene have one or more of the following variants, the presence of said nucleotide being an indicator for the individual from whom the sample has been derived responding to the pharmaceutical
   1. An Adenosine at position 635 of the genomic ADAMTS4 sequence
   2. An Adenosine at position 820 of the genomic ADAMTS4 sequence
   3. A Thymidine at position 835 of the genomic ADAMTS4 sequence
   4. A Guanosine at position 2564 of the genomic ADAMTS4 sequence
   5. A Guanosine at position 10570 of the genomic ADAMTS4 sequence
b. Either or both alleles of the ADAMTS4 gene have one or more of the following variants, the presence of which is an indicator for the individual from whom the sample has been derived responding to the pharmaceutical:
   1. A nucleotide other than Adenosine, and preferably a Guanosine at position 635 of the genomic ADAMTS4 sequence
   2. A nucleotide other than Adenosine, and preferably a Guanosine at position 820 of the genomic ADAMTS4 sequence
   3. A nucleotide other than Thymidine and preferably a Cytidine at position 835 of the genomic ADAMTS4 sequence
   4. A nucleotide other than Guanosine and preferably a Cytidine at position 2564 of the genomic ADAMTS4 sequence
   5. A nucleotide other than Guanosine and preferably an Adenosine at position 10570 of the genomic ADAMTS4 sequence
c. The amount of ADAMTS4 mRNA and/or protein in the sample is different from that in one or more comparative/reference samples, e.g. from one or more reference individuals having a known genetic background with respect to the ADAMTS4 gene (e.g. one or more of the polymorphisms according to a or b), the presence of a different amount being an indicator for the individual from whom the sample has been derived responding to the pharmaceutical.
d. The ADAMTS4 protein has one or both of the polymorphisms Ala 77, Thr 77, Pro 720 or Ala720.

With other methods and procedures for identification also being conceivable.

The determination of the type of nucleotide for the different aspects of present invention can be performed according to methods known in the art. This can e.g. be achieved by
a) providing an isolated biological sample comprising genomic DNA or providing isolated genomic DNA
b) amplifying a nucleic acid by carrying out a PCR reaction using primers able to amplify a nucleic acid comprising one or more of the positions 635, 820, 835, 2564 and/or 10570 of the genomic sequence of the ADAMTS4 gene
c) Sequencing the nucleic acid as defined in b).

Another possibility to determine the type of nucleotide is e.g. by
a) providing an isolated biological sample comprising genomic DNA or providing isolated genomic DNA
b) Immobilizing the genomic DNA on a suitable support;
c) Hybridizing to the immobilized DNA one or more probes, which, under standard conditions, are capable of binding specifically to nucleic acids having a (genomic) ADAMTS4 sequence and which have a specificity for a particular nucleotide at one or more of the positions 635, 820, 835, 2564 and or 10570 of the ADAMTS4 gene.

According to another possibility the type of nucleotide can also be determined on the basis of the above two methods, but using cDNA generated from RNA instead of using genomic DNA.

The amount of mRNA can e.g. be determined by
a. Providing a biological sample comprising mRNA or providing isolated mRNA from the sample of a);
b. Amplifying a nucleic acid by RT-PCR using primers having the ability to amplify a nucleic acid derived from the ADAMTS4 mRNA;
c. Quantifying the amount of the amplified nucleic acid and comparing it with the amount of nucleic acid amplified in at least one reference sample (i.e. positive and/or negative control samples).

The concept of positive or negative controls for verifying the result of any given analytical (biological, biochemical or chemical) reaction is well known to the skilled artisan. It comprises e.g. reactions performed in the same manner as the original analytical experiment, but lacking one or more defined components (e.g. lacking ADAMTS4 protein or mRNA or lacking a specific ADAMTS4 antibody, etc.) to discriminate specific signals which are the outcome of said experiments from so called “background” signals (artificial signals created by a given analytical method) (negative controls). It also comprises reactions performed in the same manner as the original analytical experiment but using an additional component that will result in a known signal for verifying that the reaction conditions in general work (positive control).

Another possibility of determining the amount of mRNA is e.g. by means of
a. Providing a biological sample, which comprises mRNA or providing isolated mRNA;
b. Transferring the mRNA to a suitable support;
c. Detecting and quantifying the ADAMTS4 mRNA on the support by means of at least one suitable probe;
d. Comparing with the amount of ADAMTS4 mRNA from one or more reference samples (e.g. positive and/or negative control samples).

Yet another possibility is a method comprising:
a. Providing a histological sample of the individual;
b. Detecting the amount of ADAMTS4 mRNA by way of hybridization reaction with a suitable mRNA probe, detecting and quantifying the hybridized probe;
c. Comparing the amount of ADAMTS4 mRNA with that in one or more reference samples (e.g. positive and/or negative control samples).

The determination of the amount of protein or the identification of a protein polymorphism can e.g. be achieved by
a. Providing a biological sample of the individual to be examined, which comprises protein;
b. Preferably isolating the protein from the sample of a);
c. Transferring the protein to a suitable support;
d. Detecting the protein by means of at least one antibody specific ADAMTS4 protein or specific for a certain ADAMTS4 protein polymorphism; and
e. Quantifying the signal and comparing it with the signal obtained from at least one reference sample (i.e. a negative control and/or a positive control sample).

Another possibility of determining the amount of protein or identifying a certain protein polymorphism is a method comprising:
a. Providing a histological sample of the individual;
b. Detecting the amount of ADAMTS4 protein by way of a binding reaction with a suitable ADAMTS4 antibody, detecting and quantifying said amount;
c. Comparing the amount of ADAMTS4 protein with that in one or more reference samples (e.g. positive and/or negative control samples).

The determination of certain protein polymorphisms can e.g. be achieved by using an antibody against ADAMTS4 protein
a. having detectably higher binding affinity for an ADAMTS4 protein with a Threonine at position 77 of the polypeptide chain than for an ADAMTS4 protein with another amino acid, especially with an Alanine, at position 77 or the polypeptide chain and/or
   having detectably higher binding affinity for an ADAMTS4 protein with an Alanine at position 720 of the polypeptide chain than for an ADAMTS4 protein not having an Alanine, and especially having a Proline, at position 720 of the polypeptide chain. In this connection, the term sample or taken or isolated sample refers to biological material taken from the patient. Biological material may include, inter alia: the cells or preparations or parts of a tissue or an organ or body fluids (e.g. lymph, saliva, blood, skin, connective tissue), or cells, preferably cells which are easy to remove, such as, for example, mucosal cells. Biological material of this kind may be obtained by common techniques such as taking a swab, taking a blood sample, tissue puncture or surgical techniques (e.g. biopsies). The samples are preferably histological specimens, cell preparations, cells, for example mucosal cells, cellular tissue, purified DNA, mRNA or protein or a body fluid such as saliva, lymph or blood or extracts or preparations of said samples thereof. The purification of naturally occurring molecules from cells or tissues and the preparation of cell or tissue extracts are well known to the skilled person (see also examples of the standard literature listed below). DNA/RNA or protein preparations can be obtained there from by means of common techniques.

Since ADAMTS4 polymorphisms have been identified in the present application for the first time as being connected to cardiovascular and peripheral vascular disorders, the present group of inventions related to one another also concerns the use of an ADAMTS4 protein or nucleic acid or of a functional fragment thereof for finding active substances for treating and/or preventing cardiovascular and peripheral vascular disorders.

According to one embodiment of the different aspects of present invention, ADAMTS4, the derivative or fragment thereof can be used as an isolated molecule.

In the context of this invention, the term “isolated molecule”, especially with respect to ADAMT4S, refers to ADAMTS4 nucleic acids or polypeptides or fragments thereof purified from natural sources (i.e. removed from their natural environment) as well as purified recombinant molecules (wherein the term purified comprises a partial purification as well as a complete purification). The isolation of nucleic acids is well known in the art (see also literature on standard laboratory procedures below). The isolation of proteins from natural sources or of recombinant protein is known in the art as well. A method for generating soluble, active aggrecanase from natural sources is also disclosed in JBC 1999 Vol. 274, P. 6594-6601.

The use according to present invention allows for the identification of novel substances for the prevention and/or treatment of cardiovascular and peripheral vascular disorders. The use according to present invention comprises the identification of substances with the desired characteristics as well as the further characterisation of substances already identified to be useful for the prevention and/or treatment of cardiovascular (i.e. the use according to present invention is useful for e.g. compound screening as well as compound profiling).

A substance as to be employed for the different aspects of present invention can be any biological or chemical substance or natural product extract, either purified, partially purified, synthesized or manufactured by means of biochemical or molecular biological methods.

A substance considered as being active in preventing or treating cardiovascular and peripheral vascular disorders in the sense of the different aspects of present invention can be any substance having an influence of one of the functions of ADAMTS4 or on the expression, amount or steady state level of ADAMTS4 in a biological system.

To this end, the substance can modulate any of the functions of ADAMTS4 (e.g. those as defined above or hereinbelow). ADAMTS4 protein activity can be modulated by the substance e.g. by direct interaction and interference with the function of ADAMTS4 polypeptide/protein or fragments thereof. The substance can also modulate the expression of ADAMTS4, e.g. on the level of transcription (initiation, elongation, termination), of transcript- or translate-processing (especially post-translational processing of the prepro- or pro-protein into the active form, which may also comprise c-terminal truncation of the full-length protein lacking the propeptide domain), transcript- or translate stability or translation. Moreover it can modulate the posttranslational processing, modification, protein folding etc. of ADAMTS4. The substance can exert the above effects directly or indirectly (indirectly meaning i.e. by interfering (positively or negatively) with natural signaling cascades having influence on ADAMTS4 function/protein activity/expression etc.). Endogenous inhibitors of ADAMTS4 function comprise, e.g. Tissue inhibitor of metalloproteinase-3 (TIMP-3; J Biol Chem 2001, 276: 12501-12504) or alpha-2-Macroglobuline (alpha2M; Curr Opinion in Pharmacology 2002, 2:322-329). Possible endogenous signal molecules exerting a positive effect on the Aggrecanase function of ADAMTS4 include, e.g. the cytokines (e.g. IL_{—}1, TNFalpha, IL-6, IL-17), retinoic acid and fragments of fibronectine; whereas n-3 fatty-acid seems to exert a negative influence on Aggrecanase function upon IL-1 stimulation (for an overview, see Curr Opinion in Pharmacology 2002, 2:322-329 and the references cited therein). Moreover the substance can also mimic ADAMTS4 activity (i.e. take over its function/role).

A fragment of ADAMTS4 can be any polypeptide or nucleic acid that is shorter than the corresponding wild type, e.g. shorter than homo sapiens (hs) ADAMTS4 according to the nucleic acids of SEQ ID No. 1 or 2, 16 or the polypeptide according to SEQ ID No. 3 or. A functional fragment of ADAMTS4 is any fragment (either polypeptide or nucleic acid), which exhibits at least one of the functions (as e.g. listed below) of ADAMTS4. An overview of the structure of ADAMTS4 can e.g. be gained from Curr Opinion in Pharmacology 2002, 2:322-329 or J Biol Chem 1999, Vol. 274: 23443-23450, esp. p. 23446. Examples of functional fragments comprise fragments comprising or consisting of one or more of the different domains of ADAMTS4 (the prodomain from approx. pos. 1 to pos 212; the catalytic domain from pos 213 to pos 436, the disintegrin domain from position 437 to position 520, the thrombospondin domain from position 521 to position 576, the cystein-rich domain from 577 to 685 and the spacer domain from position 686 to position 837 (see J. Biol. Chem. 2002, 277: 42775-42780), such as: the propeptide domain containing a probable cysteine switch, the catalytic domain or the thrombospondin-like motif or parts thereof involved in binding of Aggrecan GAG chains (see e.g. J. Biol Chem 2000, 275: 25791-25797, especially for peptides representing regions of the Tsp motif or J. Biol. Chem. 2002, 277: 42775-42780 for autocatalytic fragments of ADAMTS4 and peptides containing consensus GAG-binding sequences presend in the ADAMTS-4 Cystein-rich spacer domains: GSKKKFDKCM, SFRKFRYG, LRRRPWAGRK).

A derivative of ADAMTS4 or of an ADAMTS4 fragment can be any modification of an ADAMTS4 nucleic acid, polypeptide or of a fragment thereof. Derivatives comprise, e.g. modifications of the amino acid or nucleotide sequence or any other kind of modification, such as a chemical or biological modification e.g. leading to the stabilization of the polypeptide or nucleic acid (such as phosphoorothioate modifications or other kinds of modifications of the nucleic acid backbone or of exchanges of the bonds between amino acids, etc.), or enabling a specific targeting of the polypeptide or nucleic acid to certain cells or facilitating its entry into or uptake by cells (such as cell-permeant phosphopeptides, ortho coupling to cell-permeant peptide vectors, e.g. based on the antennapedia/penetratin, TAT, and signal-peptide based sequences; or coupling to parts of ligands for specific transporters or importers).

The term “functional derivative” of ADAMTS4 comprises any kind of modification of ADAMTS4 with respect to the naturally occurring form (either polypeptide or nucleic acid), which at least has one of the functions of ADAMTS4. Present invention also comprises functional derivatives of fragments of ADAMTS4.

Functions of ADAMTS4 comprise the functions described above, for example the ability of an ADAMTS4 protein or protein fragment to bind and proteolytically cleave other proteins or protein-fragments, e.g. to bind and/or to cleave Aggrecan (Science 1999, 284: 1664-1666) or fragments thereof being able to interact or be cleaved by ADAMTS4 (i.e. Aggrecan fragments comprising the portion from residues Glu373 to Ala 374 containing the interglobular domain (IGD) and/or comprising one or more of four ADAMTS4-cleavage sites in the chondroitin-sulfate-rich region between the globular (G) domains 2 and 3 (G2 and G3) or comprising the whole chondroitin-sulfate-rich region and or comprising the Glycosaminoglycan-rich C-terminal region (the GAG region)), Brevican (J. Biol Chem 2000, 275: 22695-22703) or fragments thereof being able to interact or be cleaved by ADAMTS4, Versican (J. Biol Chem 2001, 276: 13372-13378) or fragments thereof being able to interact or be cleaved by ADAMTS4, to interact and/or be inhibited by tissue inhibitor of metalloproteinase 3 (TIMP-3, J Biol Chem 2001, 276: 12501-12504) or to bind and/or cleave alpha-2-Macroglobulin (alpha2M; Curr Opinion in Pharmacol 2: 322-329). With regard to ADAMTS4 nucleic acids or fragments thereof, ADAMTS4-functions comprise for example the ability to interact with other molecules, such as, for example, specific hybridization primers or probes, the ability to control transcription of a downstream coding sequence, to code for ADAMTS4 protein, etc.). Functions of ADAMTS4 comprise also generally the ability of ADAMTS4 (protein or nucleic acid) or fragments thereof to interact with other molecules (comprising, but not limited to, proteins or protein fragments (i.e, with Aggrecan, Brevican, Versican or fragments thereof), nucleic acids (i.e. ADAMTS4 nucleic acids to specifically hybridise with other nucleic acids), synthetic molecules (i.e. ADAMTS4 protein or fragments to specifically interact with synthetic drugs). For the functions listed above, see also Curr Opinion in Pharmacol 2: 322-329, especially page 322, right column, second paragraph to first paragraph on page 323 and page 323, right column, second paragraph to second paragraph, right column, page 324.

The identification of active substances can e.g. be performed by means of one or more of the methods identified hereinbelow, such as:

A method for identifying substances active in preventing or treating cardiovascular and peripheral vascular disorders comprising:
a. Contacting a ADAMTS4 protein or functional fragment or derivative thereof with a test substance; and
b. Determining whether the test substance modulates the activity of the ADAMTS4 protein or functional fragment or derivative thereof.

A method for identifying substances active in preventing or treating cardiovascular and peripheral vascular disorders comprising:
a. Contacting a cell, which has a detectable amount or activity of ADAMTS4 or of a functional fragment or derivative thereof, with a test substance;
b. Determining whether the test substance is able to modulate the amount or activity of ADAMTS4 or the functional fragment or derivative thereof present in the cell.

A substance/test substance/active substance as to be employed for the different aspects of present invention can be any biological or chemical substance or natural product extract, either purified, partially purified, synthesized or manufactured by means of biochemical or molecular biological methods.

Wherein a substance able to detectably modulate the ADAMTS4 amount or activity is considered a substance active in preventing or treating cardiovascular and peripheral vascular disorders. The detectable amount of ADAMTS4 can either refer to a detectable amount ADAMTS4 nucleic acid (mRNA, cDNA or genomic DNA) and/or protein (prepro/pro/ripe protein). The detectable activity can either refer to transcriptional and/or translational and/or protein activity of ADAMTS4 DNA/mRNA or protein.

Within the different aspects and embodiments of present invention the term modulation refers to activation or an inhibition.

Another example is a method for identifying substances active in preventing or treating cardiovascular and peripheral vascular disorders comprising:
a. Contacting a nucleic acid coding for an ADAMTS4 protein or a functional fragment or derivative thereof with a test substance in a transcriptionally active system;
b. Determining the amount of mRNA coding for the ADAMTS4 protein or functional fragment or derivative present in said system in presence of said substance;
c. Determining the amount of mRNA coding for the ADAMT4 protein or functional fragment or derivative present in said system in the absence of said substance;
d. Determining whether the substance is capable of modulating the amount of mRNA coding for the ADAMTS4 protein or functional fragment or derivative present in said system.

Wherein a substance capable of modulating the amount of ADAMTS4 mRNA present in said system is considered a substance active in preventing or treating cardiovascular and peripheral vascular disorders.

A transcriptionally active system is any biochemical or cellular system, which at least has the ability to perform a transcription reaction of a transcription unit. Such systems are well known in the art and comprise cells (e.g. usual laboratory strains or cell lines as well as primary cultures of eucaryotic or prokaryotic cells) as well as in vitro transcription systems or kits (e.g. on basis of cell extracts) which are also commercially available. In case of present invention this can be a biochemical or cellular system expressing ADAMTS4 mRNA (e.g. the mRNA according to NM_{—}005099.3) or expressing mRNA coding for a functional ADAMTS4 fragment.

The determination of the mRNA amount present in the system can be performed according to techniques well known in the state of the art (etc. direct labelling of the product by means of radioactive or fluorescent labelling or product detection by use of specific primers or probes etc.).

Another example is a method for identifying substances active in preventing or treating cardiovascular and peripheral vascular disorders comprising:
a. Contacting a nucleic acid coding for an ADAMTS4 protein or a functional fragment or derivative thereof with a test substance in a translationally active system;
b. Determining the amount of ADAMTS4 protein or functional fragment or derivative thereof present in said system in presence of said substance;
c. Determining the amount of ADAMTS4 protein or functional fragment or derivative thereof present in said system in the absence of said substance;
d. Determining whether the substance is capable of modulating the amount of ADAMTS4 protein or functional fragment or derivative thereof present in said system.

Wherein a substance capable of modulating the amount of ADAMTS4 protein, derivative or fragment present in said system is considered to be a substance active in the prevention or treatment of cardiovascular and peripheral vascular disorders.

A translationally active system is any biochemical or cellular system, which at least has the ability to perform a translation reaction of a transcript. Such systems are well known in the art and comprise cells (e.g. usual laboratory strains or cell lines as well as primary cultures of eucaryotic or prokaryotic cells) as well as in vitro translation systems (which are also commercially available, e.g. as kits). For the in vitro translation of a nucleic acid, the nucleic acid is subcloned in a suitable vector, followed by the expression of the polypeptide in suitable buffers and cell extracts (e.g. reticulocyte lysate). Vectors, necessary reagents and protocols with suitable conditions are known in the art and commercially available.

In the context of present invention, the term “polypeptide” refers to a molecule comprising amino acids bound to each other by peptide bonds and which contain at least 10 amino acids coupled to each other in a linear mode. Shorter molecules of this kind are referred to as peptides. The term “protein” refers to molecules comprising at least one polypeptide chain but can also refer to molecules comprising two or more polypeptide chains associated or bound to each other. Thus, the term “protein” comprises the term “polypeptide”.

The detection of the ADAMTS4 protein present in said system can be performed according to techniques well known in the art (e.g. direct radioactive or fluorescent labelling of the translate or the employment of specific antibodies, tagging of the protein and detection of the tag, etc.).

Another example concerns a method for identifying substances active in preventing or treating cardiovascular and peripheral vascular disorders comprising:
a. Providing a cell transfected with a nucleic acid vector comprising the promotor of an ADAMTS4 gene or a functional fragment thereof operationally coupled to a reporter gene or a functional fragment thereof:
b. Providing a cell transfected with a control vector which comprises a reporter gene or a functional fragment thereof not being operationally coupled do a functional ADAMTS4 promotor;
c. Determining the reporter gene activity of the cell according to a) and b) in the presence of a test substance;
d. Determining the reporter gene activity of the cell according to a) and b) in absence of the test substance.

Wherein a substance capable of significantly modulating (i.e. increasing or decreasing) reporter gene activity according to a) without significantly modulating reporter gene activity of b) (i.e. capable of specifically increasing ADAMTS4 promoter activity) is considered to be a substance active in the prevention or treatment of cardiovascular and peripheral vascular disorders.

A significant modulation is any modulation (i.e. increase or decrease) higher than the standard deviation; preferably it is at least two times as high as the standard deviation.

The above aspect of present invention is based on a typical reporter gene assay commonly known in the art. To this end, the promoter of choice is inserted into an expression vector suitable for the type of host cell chosen, upstream of the reporter gene of choice in such a way as to allow for an expression of the reporter gene if the promoter is active. The construct is subsequently introduced into the host cell of choice. Suitable methods for transformation or transfection are well known in the art as well as conditions for cell cultivation and detection of reporter gene expression (see e.g. standard literature listed below). Suitable conditions are well known in the art as well as vectors, reporter genes and necessary reagents, which are also commercially available.

A vector is a circular or linear nucleic acid molecule, e.g. a DNA plasmid, bacteriophage or cosmid, by aid of which nucleic acid fragments (e.g. cut out from other vectors or amplified by PCR and inserted in the cloning vector) can specifically be amplified in suitable cells or organisms. Expression vectors enable the heterologous expression of a gene of interest (e.g. a reporter gene), in the host cell or organism. The type of cell or organism largely depends on the aim and the choice lies within the knowledge of the skilled artisan. Suitable organisms for the amplification of a nucleic acid are e.g. mostly single cell organisms with high proliferation rates, like e.g. bacteria or yeast. Suitable organisms can also be cells isolated and cultivated from multicellular tissues, like e.g. cell lines generated from diverse organisms (e.g. SF9 cells from Spodoptera Frugiperda, etc.). Suitable cloning vectors are known in the art and commercially available at diverse biotech suppliers like, e.g. Roche Diagnostics, New England Biolabs, Promega, Stratagene and many more. Suitable cell lines are e.g. commercially available at the American Type Culture Collection (ATCC).

For the heterologous expression of a protein or polypeptide, the cell can be any prokaryotic or eucaryotic cell suitable for transfection with a nucleic acid vector and of expressing the gene of interest, e.g. a reporter gene. Possible examples thereof are primary cells or cultured cells, preferably eukaryotic cell cultures, which have originally been obtained, for example, from multicellular organisms or tissues (such as, for example, HeLA, CHO, COS, SF9 or 3T3) or which themselves are unicellular organisms, such as, for example, yeast cells (e.g. S. pombe or S. cerevisiae) or prokaryotic cell cultures, or Pichia or E. coli. Cells and samples from tissues may be obtained by known techniques of the prior art (e.g. taking blood samples, tissue puncture or surgical techniques). Suitable for use for the inventive use of ADAMTS4 are also isolated cells which naturally produce and, where appropriate, secrete ADAMTS4, such as, for example, endocrine K cells, it being possible to determine directly the ability of a cardiovascular pharmaceutical to increase or decrease the amount of ADAMTS4 produced.

In the context of the present application, the term “transfection” refers to the introduction of a nucleic acid vector into a (pro- or eukaryotic) host cell and thus includes the term “transformation”. Said transfection may be stable or transient and can be carried out on the basis of common methods.

The ADAMTS4 promoter region is the part of the ADAMTS4 gene, which is capable of controlling transcription of a gene product of interest, if the coding sequence of the gene of interest is cloned into a suitable vector, functionally downstream of the promoter/enhancer, and is transfected into a suitable host cell. Functional fragments of the ADAMTS4 promoter are ADAMTS4 promoter fragments, which, under the conditions given, can likewise control the transcription of downstream coding sequences. The identification of suitable fragments lies in the skill of the responsible artisan.

A reporter gene can be any gene that allows for an easy quantification of its gene product. A vast variety of reporter genes for eukaryotic or prokaryotic hosts as well as detection methods and necessary reagents are known in the art and commercially available. These comprise e.g. the genes of beta Lactamase (lacZ), Luciferase, Green or Blue fluorescent protein (GFP or BFP), DsRed, HIS3, URA3, TRP1 or LEU2 or beta Galactosidase. These genes encode proteins, which can be easily detected by means of a visible (colour or luminescent) reaction (e.g. lacZ, Luciferase). These comprise gene-products which can be easily detected by means of a visible (colour or luminescent) reaction or gene-products conferring resistance towards antibiotics like Ampicillin or Kanamycin when expressed. Other reporter gene-products enable the expressing cells to grow under certain conditions like e.g. auxotrophic genes.

A functional fragment of a reporter gene is any fragment of a given reporter gene that allows for an easy quantification of its gene product.

Within the context of the above aspect of present invention the control vector can be any suitable vector which comprises a reporter gene or functional fragment thereof, but wherein reporter gene expression is not driven by a (functional) ADAMTS4 promoter. This can e.g. mean that the reporter gene or functional fragment thereof is not operationally coupled to a functional ADAMTS4 promoter (i.e. either totally devoid of an ADAMTS4 promoter, comprises a non functional ADAMTS4 promoter or promoter fragment or wherein the coupling of promoter and reporter gene is not functional). This can also mean that the reporter gene or functional fragment thereof is operationally coupled to another promoter than the ADAMTS4 promoter (e.g. SV40 or another standard promoter). The functional vector and the control vector can also be transfected to the same cell, but in which case the reporter genes need to be different.

Another aspect of present invention concerns a high throughput screen based on a method according to one of the above novel methods for the identification of active substances (such methods are also called “assays”).

Analytical methods or analytical systems, so-called assays, which are used to measure the activity or concentration of defined target molecules (so-called targets, mostly proteins or nucleic acids) as parameter for the effectiveness of a potential pharmaceutical compound, are well known in the state of the art. Assays comprise for example biochemical analytical methods or systems using isolated or partly isolated components that are put together to a reaction mixture within a defined space and time, in which the effectiveness of the potential pharmaceutical compounds can be tested (e.g. the above methods). For measuring protease activity, these encompass, e.g. radioisotopic or fluorescent assays for measuring the interaction of a labelled member with a non-labelled member (e.g. the interaction of a labelled substrate and an unlabeled protease, wherein upon cleavage of the substrate part of the labelled substrate is set free; thus the protease activity can be measured by detecting and quantifying the amount of the labelled member set free in a given time). Other examples of assays comprise cell based assays (e.g. the above reporter assays or phenotypical assays, wherein the activity of a substance can be monitored by a change in the phenotype of a cell).

Different types of assays are commonly known in the state of the art and commercially available from commercial suppliers.

According to a further embodiment of the different aspects of present invention, a ADAMTS4 nucleic acid is used which comprises sequences comprising one or more of the positions 635, 820, 835, 2564 and 10570 or an ADAMTS4 polypeptide is used which comprises one or both of the positions 77 and 720.

Since the ADAMTS4 gene and protein variants identified, as being most significantly connected with the onset of cardiovascular and peripheral vascular disorders are G635A, A635A, G820A, A820A, C835T, C2564G, G2564G, A10570G and G10570G, Thr77 and Ala 720, a preferred embodiment of the present invention relates to the use of one or more of the above polymorphisms or of an ADAMTS4 nucleic acid or polypeptide having one or more of the above polymorphisms, e.g. for performing one or more of the methods according to present invention.

Normally, individual SNPs in the wild type sequence of the gene to be studied are not taken into account in the screening for active compounds by using target genes (“molecular targets”). Since the present application has identified ADAMTS4 variants as being connected with the occurrence of cardiovascular and peripheral vascular disorders, the use of variants of this kind (in particular against the background of the particular physiological makeup of the cells, which accompanies this) should produce a higher probability of finding active compounds suitable for treating and/or preventing cardiovascular and peripheral vascular disorders. Since in fact, individuals having a genetic and physiological makeup of this kind (e.g. having one or more of G635A, A635A, G820A, A820A, C835T, C2564G, G2564G, A10570G and G10570G, Thr77 and Ala 720) also are more likely to suffer from cardiovascular and peripheral vascular disorders, the majority of the active compounds found herein targets precisely this patient group. On the other hand, the use of the ADAMTS4 G635G, G820G, C835C, C2564C, A10570A, Ala77 and Pro720 variants should result in finding active compounds to which in particular patients having this gene or protein variant respond. The use of the ADAMTS4 G635G, G820G, C835C, C2564C, A10570A, Ala77 and Pro720 variants therefore corresponds to another embodiment of present invention.

In addition, the SNPs in the ADAMTS4 gene are suitable for use in active compound screening using targets other than ADAMTS4 itself: thus it is possible to use cells in cellular assays for finding active compounds for the treatment and/or prevention of cardiovascular and peripheral vascular disorders, which compounds have the ability to influence the function and/or activity and/or amount of a target other than ADAMTS4, specifically cells whose genome has a defined variant of the ADAMTS4 gene with respect to positions 635, 820, 835, 2564 and/or 10570. In this way it is possible to screen specifically for compounds active in preventing or treating cardiovascular and peripheral vascular disorders, even those that intervene in the function of a gene other than the mutated one, against the genetic background, which is preferably connected with the disease to be treated.

A further aspect of the present invention relates to the use of means for the detection of ADAMTS4 for diagnosing cardiovascular and peripheral vascular disorders or a predisposition for cardiovascular and peripheral vascular disorders by analyzing a biological sample taken from the body of an individual to be examined.

In this connection, the presence of one or more of the following variants preferably indicates an increased risk: G635A, A635A, G820A, A820A, C835T, C2564G, G2564G, A10570G, G10570G, Thr77, Ala720.

A means for detecting ADAMTS4 may be any means suitable for detecting an ADAMTS4 protein or nucleic acid in a biological sample.

The means for detection may be, for example, a means for detecting ADAMTS4 mRNA or protein in the sample; e.g. a means on the basis of which the amount of ADAMTS4 mRNA or protein in a sample can be quantified, (for example suitable primers, probes, anti-ADAMTS4 antibodies, etc.; for example, a nucleic acid probe which is able to hybridize under standard conditions specifically to ADAMTS4 mRNA or cDNA, for example for use in a Northern blot or in micro arrays or a Primer set for quantitative Reverse Transcriptase (RT) Polymerase Chain Reaction (PCR)). Another example relates to means for determining the type of nucleotide at one or more of the positions 635, 820, 835, 2564 and 10570 of the ADAMTS4 gene, for example a suitable PCR primer set (e.g. genomic or cDNA Primers) to be used e.g. in PCR Sequencing, a probe (to be used, e.g. in Southern Blots or Chip- or Array Hybridisation) or a specific anti-DNA antibody to be used, e.g in immuno(histo)chemical, -fluorescent or -radiochemical techniques known in the art. Yet another example relates to a means for determining the type of amino acid present at one or both of the positions 77 and 720 within the ADAMTS4 protein, e.g. an antibody specific for a certain protein polymorphism.

According to one embodiment, the means for detection is a means for detecting the amount of ADAMTS4 mRNA in a biological sample, such as, for example one or more of the primers according to SEQ ID No. 5, 6, 8, 9, 11, 12, 14 and 15.

According to another embodiment, the means for detection is a means for determining the type of nucleotide at one or more of the positions 635, 820, 835, 2564 and 10570 of the ADAMTS4 gene, such as, for example one or more of the primers according to SEQ ID No. 5, 6, 8, 9, 11, 12, 14 and 15.

The design and synthesis of suitable primers is known in the prior art; such primers may also be obtained commercially. According to a preferred embodiment, these are the primers according to SEQ ID Nos, 5, 6, 8, 9, 11, 12, 14 and 15. Nucleic acids are sequenced by means of conventional routine methods, for example by using customary laboratory robots, which are sold, for example, by companies such as Life Technologies, Applied Biosystems, BioRad, etc.

The design and preparation of suitable probes are likewise known in the prior art (see, for example, the standard literature listed).

In a further preferred embodiment of one of the uses or these methods according to the invention, the change in the amount of protein or the determination of a given protein-polymorphism within the ADAMTS4 protein is determined with the aid of at least one antibody. Preferred detection methods here are ELISA, Western blot, protein chip and spectrometric methods.

The preparation of suitable antibodies or functional fragments thereof is known in the prior art, for example by immunizing a mammal, for example a rabbit, with ADAMTS4 protein or a fragment thereof, where appropriate in the presence of a suitable adjuvant (Freund's adjuvant or aluminum hydroxide gel, see, for example, Diamond, B. A. et al. (1981) The New England Journal of Medicine: 1344-1349). The polyclonal antibodies produced in the animal as a result of the immunological reaction may subsequently be isolated and purified by means of known methods, for example by column chromatography. Monoclonal antibodies may be obtained, for example, according to the known method by Winter and Milstein (Winter, G. & Milstein, C. (1991) Nature, 349, 293-299). Suitable methods for preparing and purifying monoclonal antibodies are known in the prior art (see standard literature). Examples of known antibodies for detecting ADAMTS4 are: Santa Cruz Biotechnology Inc. Cat. No.: sc-25582, sc-16534 or Chemicon International Cat. No.: AB19166, AB19165 or ABCAM Cat. No.: ab11567 or Affinity Bioreagents Cat. No.: PA1-1750.

In the context of the present group of related inventions, the term antibody or antibody fragment also refers to recombinantly produced antibodies or antigen binding sites thereof, which may also be modified, where appropriate, such as, for example, chimeric antibodies, humanized antibodies, multifunctional antibodies, bi- or oligospecific antibodies or F(ab) or F(ab)₂ fragments (see, for example, EP-B1-0 368 684, U.S. Pat. No. 4,816,567, U.S. Pat. No. 4,816,397, WO 88/01649, WO 93/06213 or WO 98/24884).

Conventional immunochemical or immunoradiological methods for detecting an antibody reaction are well known to the skilled worker. Common methods are based, for example, on binding of a specific primary antibody to the antigen to be identified, binding of a secondary antibody, which usually recognizes species-specific epitopes on said primary antibody. Binding of said secondary antibody is utilized here in order to generate a detectable signal (for example a radioactive signal, when radio labeled secondary antibodies are used, or a fluorescent signal, when fluorescence-coupled secondary antibodies are used, or a colorimetrically determinable signal, for example when enzyme-coupled secondary antibodies are used, etc.), see also the literature listed below regarding standard methods.

The present group of inventions related to one another additionally also relates to a diagnostic kit for detecting a predisposition for cardiovascular and peripheral vascular disorders, which kit comprises at least one means for detecting ADAMTS4 in biological samples.

In the context of the present invention, the term “kit” (kit of parts) means any combination of components identified herein which have been combined to give a spatially and functionally connected unit for performing a given task (here e.g. diagnosis of cardiovascular and/or peripheral vascular disorders or a predisposition therefore) which may additionally comprise further parts.

A diagnostic kit according to the present invention comprises at least one means for detecting ADAMTS4 in a biological sample. Suitably, it may furthermore include suitable buffers and/or further reagents for detecting ADAMTS4 and/or for preparing or methoding samples and also, where appropriate, instructions for carrying out the particular detection method.

According to a preferred embodiment of the uses or methods according to the invention, the presence of one or more of the variations in the ADAMTS4 gene are detected by PCR and, where appropriate, subsequent sequencing or by using a genomic nucleic acid probe.

Suitable protocols and reagents for PCR or hybridization with suitable probes, which bind, for example, immobilized genomic DNA on suitable supports (e.g. membranes or chips) are well known in the prior art.

A nucleic acid molecule may “hybridize” with another one, if single-stranded forms of both molecules can attach to one another under suitable reaction conditions (temperature and ion concentration of the surrounding medium) in order to form a new double-stranded nucleic acid molecule. In order to hybridize, the nucleic acid molecules attaching to one another must have complementary sequences. However, depending on the chosen stringency conditions, base mismatches are also possible, without stopping an attachment. The term “stringency” describes reaction-conditions, which influence the specificity of hybridization, when two single-stranded nucleic acid molecules attach to one another, and thus also determine how many mismatches or how strong a mismatch between the two molecules are tolerated during attachment. The stringency and thus also the specificity of a reaction here depends inter alia on the temperature and the buffer conditions. Adequate stringency-conditions for attaching two given nucleic acid molecules also depend on the length, the type of nucleic acid molecules and the degree of complementarity. Said parameters and the determination of suitable conditions for a given analytical method are well known to the skilled person and may also be found in the literature of standard laboratory methods (e.g. “Current Protocols in Molecular Biology”, John Wiley & Sons, N.Y. (1989), 6.3.1-6.3.6).

According to a preferred embodiment of the present group of inventions related to one another, the individual is a patient having glucose metabolism disorders, preferably a diabetes patient and, particularly preferably, a patient having type II diabetes. In addition, the individual may also be hypertensive and/or may have already suffered a myocardial infarction. The cardiovascular disease is preferably a coronary heart disease (>50% stenosis), a coronary artery disease, myocardial infarction, premature myocardial infarction, acute coronary syndrome or angina pectoris (in particular instable angina).

The isolated sample used for the methods, use or test kit of the present invention is preferably a human sample and the individual to be examined is preferably a human being. Said sample may be in particular: a histological sample, a biopsy sample, a cell (e.g. mucosal cells), a cell extract, cellular tissue, body fluid, preferably blood, saliva, lymph or urine.

The invention additionally relates to an isolated ADAMTS4 nucleic acid or a fragment thereof sequence according to SEQ ID No. 1, 2 or 16 and comprising one or more of the following SNPs:
a. An Adenosine at position 635 of the genomic ADAMTS4 sequence,
b. An Adenosine at position 820 of the genomic ADAMTS4 sequence,
c. A Thymidine at position 835 of the genomic ADAMTS4 sequence,
d. A Guanosine at position 2564 of the genomic ADAMTS4 sequence and/or
e. A Guanosine at position 10570 of the genomic ADAMTS4 sequence.

The invention moreover relates to an isolated ADAMTS4 protein having one or both of the following protein polymorphisms:
a. A Threonine at position 77 of the polypeptide chain of the ADAMTS4 protein;
b. An Alanine at position 720 of the polypeptide chain of the ADAMTS4 protein;

An isolated ADAMTS4 protein according to claim 90 having the sequence according to one of the SEQ ID No. 3, 17 or 18.

Another aspect of the invention concerns one or more of the primers or primer sets according to SEQ ID Nos. 5, 6, 8, 9, 11, 12, 14 and 15 or one or more of the nucleic acids according to SEQ ID Nos. 4, 7, 10 or 13 (which e.g. can serve as nucleic acid probes).

The invention will be illustrated in more detail below on the basis of examples, which are not to be regarded as limitation, in combination with the figures and tables:

### REFERENCES

Standard Literature of Laboratory Methods:

(Unless stated otherwise, the laboratory methods mentioned herein are or can be carried out according to the standard literature listed below.)

Sambrook et al. (1989) Molecular Cloning: A Laboratory Manual. Second edition. Cold Spring Harbor Laboratory Press, Cold Spring Harbor, N.Y. 545 pp;

Current Protocols in Molecular Biology; regularly updated, e.g. Volume 2000; Wiley & Sons, Inc; Editors: Fred M. Ausubel, Roger Brent, Robert Eg. Kingston, David D. Moore, J. G. Seidman, John A. Smith, Kevin Struhl.

Current Protocols in Human Genetics; regularly updated; Wiley & Sons, Inc; Editors: Nicholas C. Dracopoli, Honathan L. Haines, Bruce R. Korf, Cynthia C. Morton, Christine E. Seidman, J. G. Seigman, Douglas R. Smith.

Current Protocols in Protein Science; regularly updated; Wiley & Sons, Inc; Editors: John E. Coligan, Ben M. Dunn, Hidde L. Plögh, David W. Speicher, Paul T. Wingfield.

Molecular Biology of the Cell; third edition; Alberts, B., Bray, D., Lewis, J., Raff, M., Roberts, K., Watson, J. D.; Garland Publishing, Inc. New York & London, 1994;

Short Protocols in Molecular Biology, 5th edition, by Frederick M. Ausubel (Editor), Roger Brent (Editor), Robert E. Kingston (Editor), David D. Moore (Editor), J. G. Seidman (Editor), John A. Smith (Editor), Kevin Struhl (Editor), October 2002, John Wiley & Sons, Inc., New York

Transgenic Animal Technology A Laboratory Handbook. C. A. Pinkert, editor; Academic Press Inc., San Diego, Calif., 1994 (ISBN: 0125571658)

Gene targeting: A Practical Approach, 2^{nd} Ed., Joyner A L, ed. 2000. IRL Press at Oxford University Press, New York;

Manipulating the Mouse Embryo: A Laboratory Manual. Nagy, A, Gertsenstein, M., Vintersten, K., Behringer, R., 2003, Cold Spring Harbor Press, New York;

Literature concerning ADAMTS4:

Prediction of the Coding Sequences of Unidentified Human Genes. X. The Complete Sequences of 100 New cDNA Clones from Brain Which Can Code for Large Proteins in vitro. Ishikawa, K., Nagase, T., Suyama, M., Miyajima, N., Tanaka, A., Kotani, H., Nomura, N. and Ohara, O. DNA Res. 5: 169-176, 1998.

ADAMTS: a novel family of extracellular matrix proteases. Tang, B. L., Int J. Biochem. Cell Biol. 33(1): 33-44, 2001.

Aggrecanase-mediated cartilage degradation. Arner, E. C., Curr Opinion in Pharmacol 2: 322-329, 2002.

Brain-enriched Hyaluronan Binding (BEHAB)/Brevican Cleavage in a Glioma Cell Line Is Mediated by a Disintegrin and Metalloproteinase with Thrombospondin Motifs (ADAMTS) Family Member. Matthews, R. T., Gary, S. C., Zerillo, C., Pratta, M., Solomon, K., Arner, E. C. and Hockfield, S. J. Biol. Chem 2000, 275: 22695-22703

Generation and Characterization of Aggrecanase, Amer, E. C., Pratta, M. A., Trzaskos, J. M., Decicco, C. P., Tortorella, M. D., J. Biol. Chemistry, 1999, Vol. 274, No. 10, p. 6594-6601;

Altered Proteolytic Activities of ADAMTS-4 Expressed by C-terminal Processing; Kashiwagi, M., Enghilöd, J. J., Gendron, C., Hughes, C., Caterson, B., Itoh, Y., Nagase, H., 2004, J. Biol. Chemistry, Vol, 279, No.11, p. 10109-10119;

Induction of Aggrecanase 1 (ADAMTS4) by Interleukin-1 Occurs Through Activation of Constitutively Produced Protein; Pratta, M. A., Scherle, P. A., Yang, G., Lui, R.-Q., Newton, R. C., 2003, Arthritis & Rheumatism; Vol. 48, p. 119-133;

### LEGEND OF THE FIGURES AND TABLES

Figures:

FIG. 1: Nucleic acid sequences of part of human ADAMTS4 gene) with the NCBI Number NM_{—}005099.2 (SEQ ID NO.1).

FIG. 2: (2a) Coding sequence of human ADAMTS4 with the NCBI Number AY044847.1 (SEQ ID No.2) and (2b) mRNA sequence of human ADAMTS4 with the NCBI Number NM_{—}005099.3 (SEQ ID No.16)

FIG. 3:

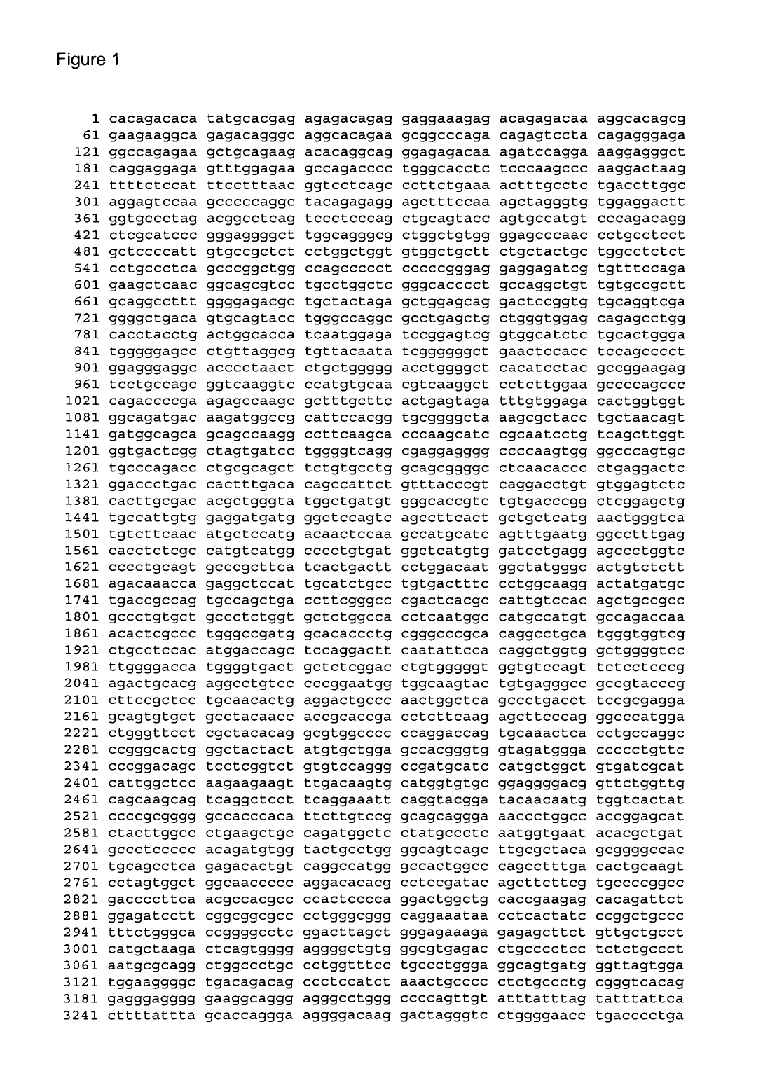

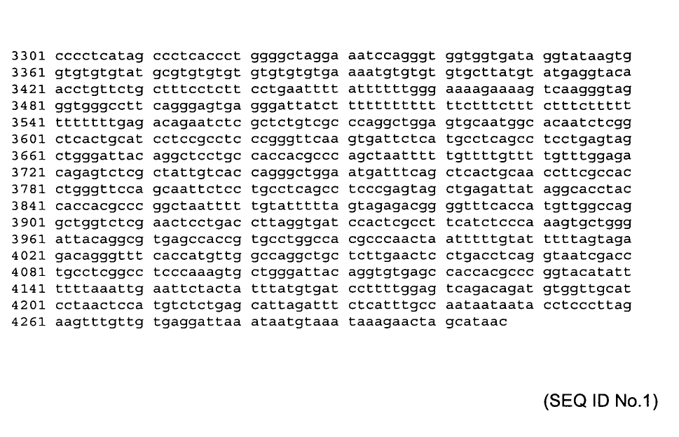

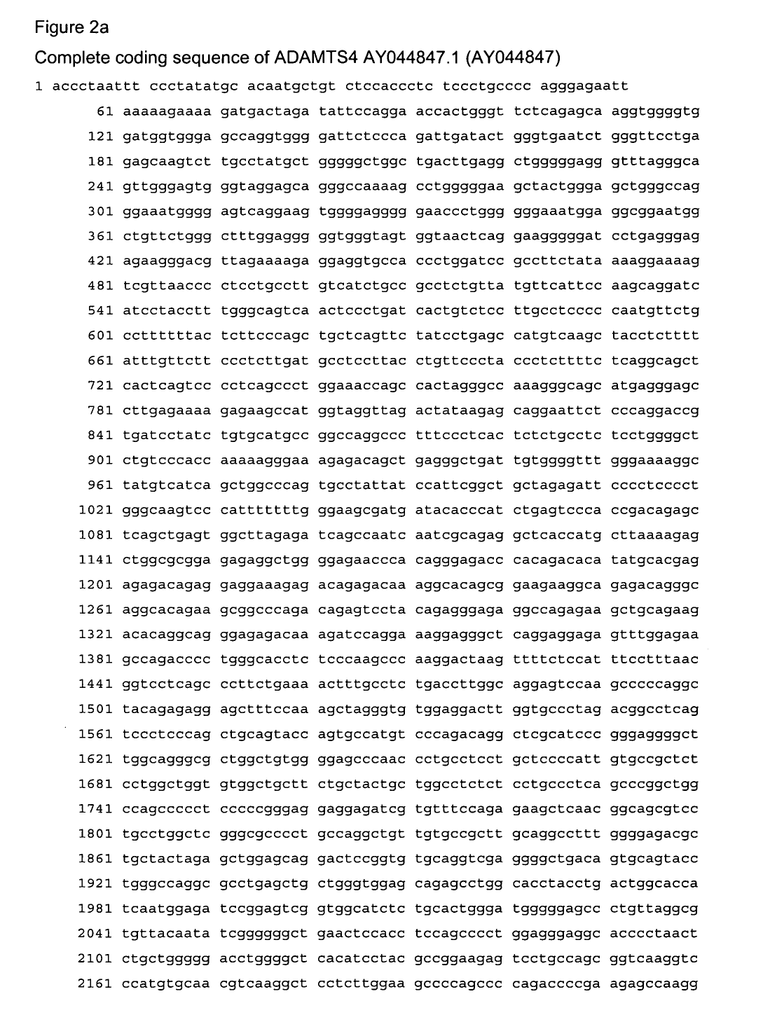

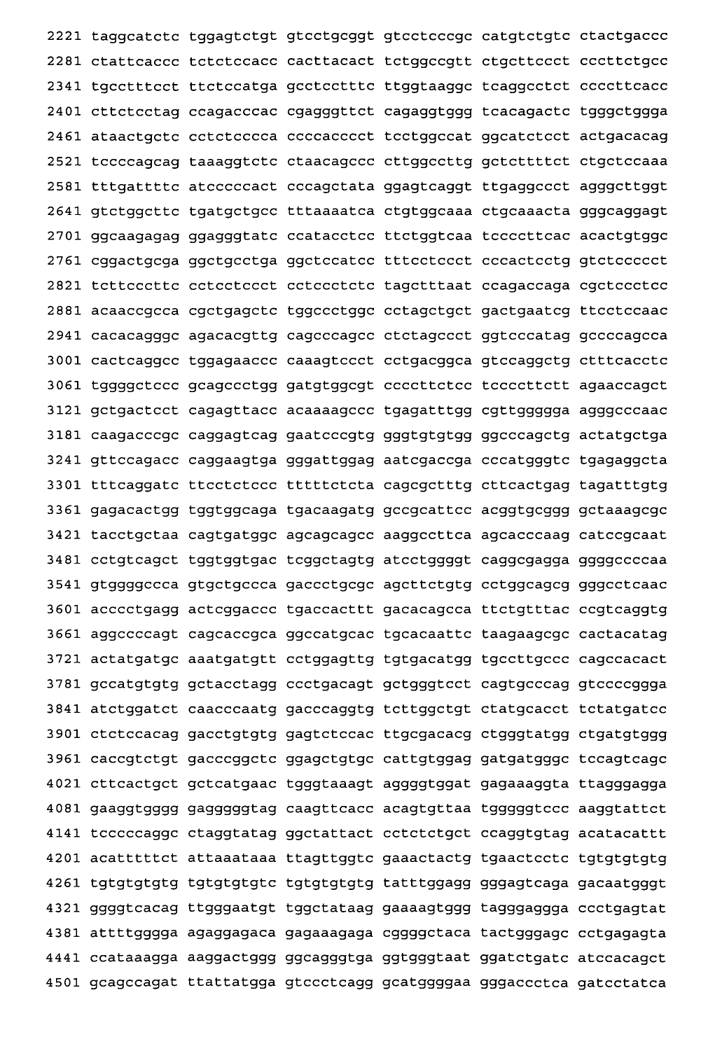

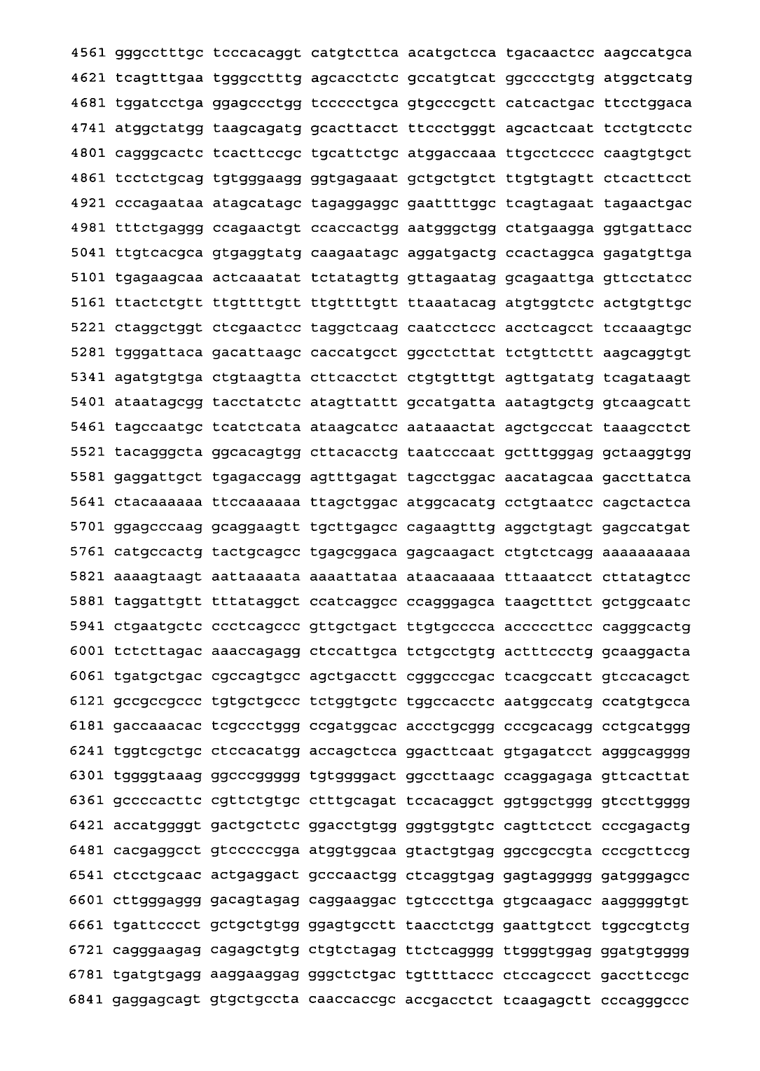

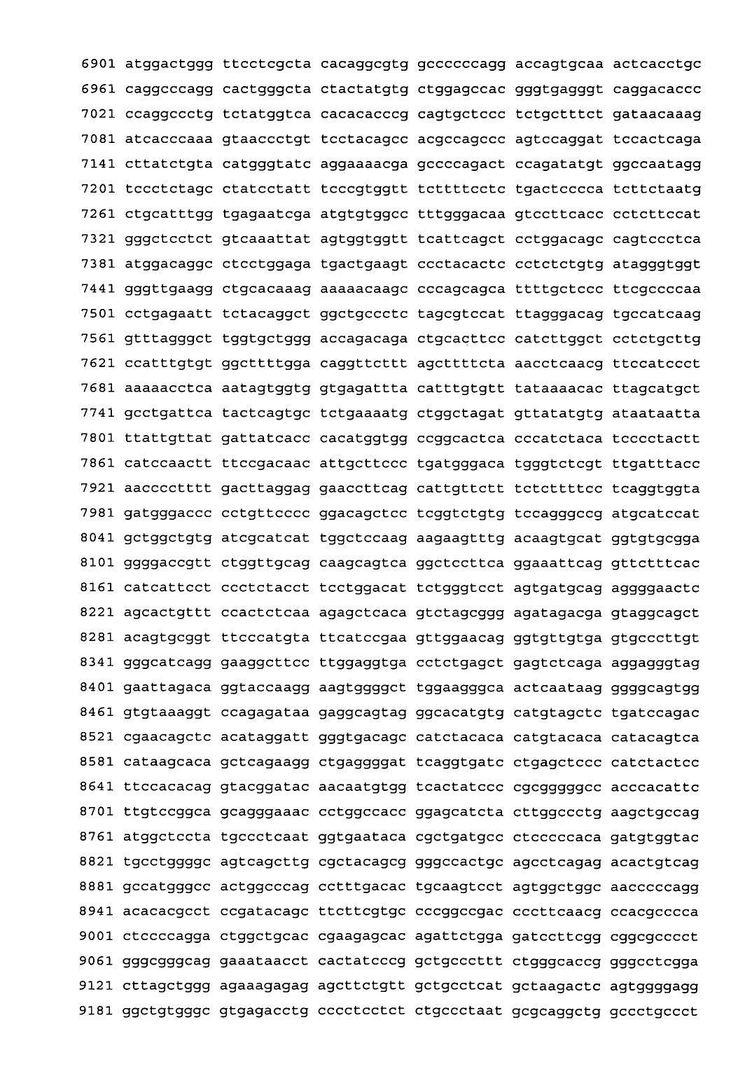

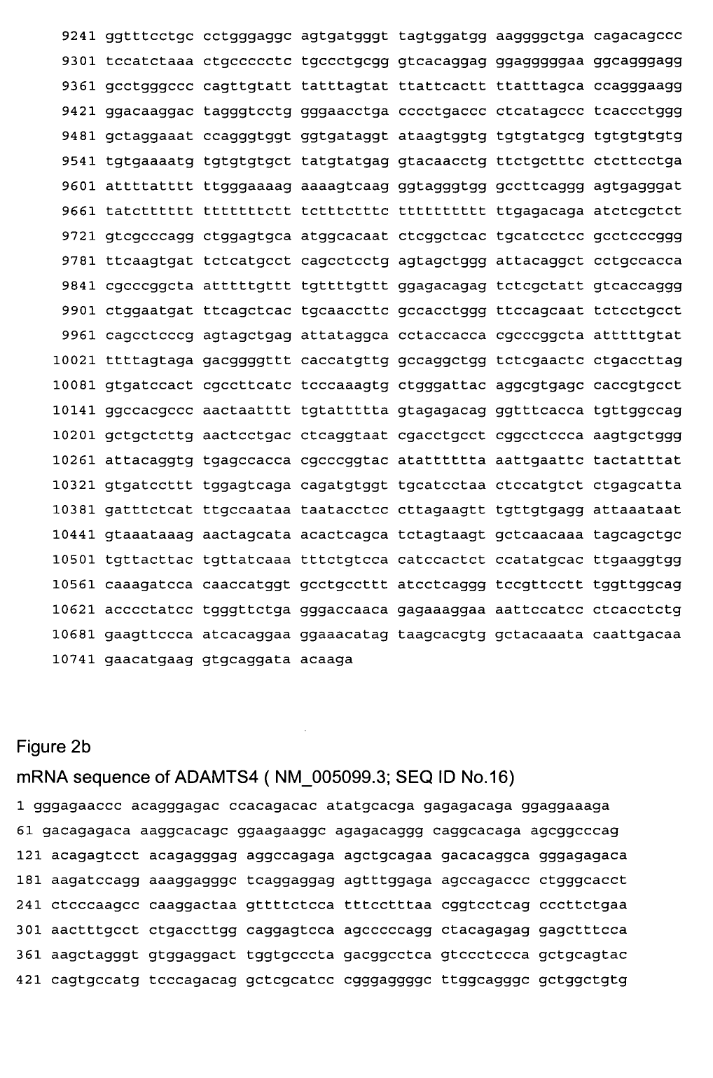

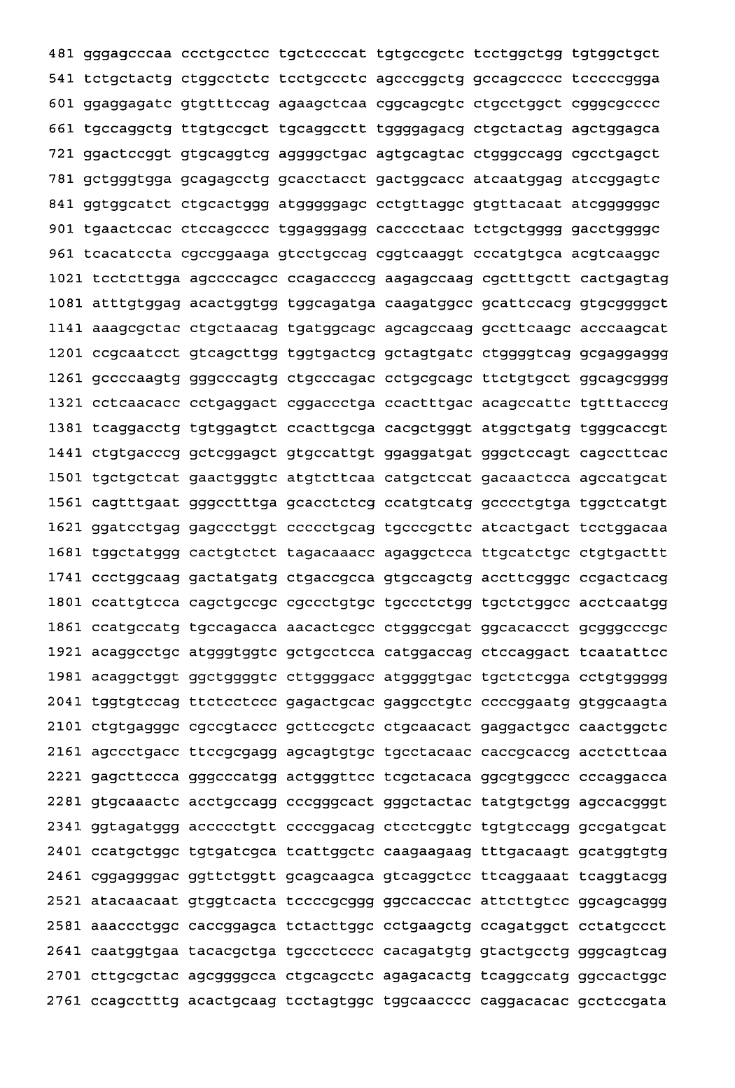

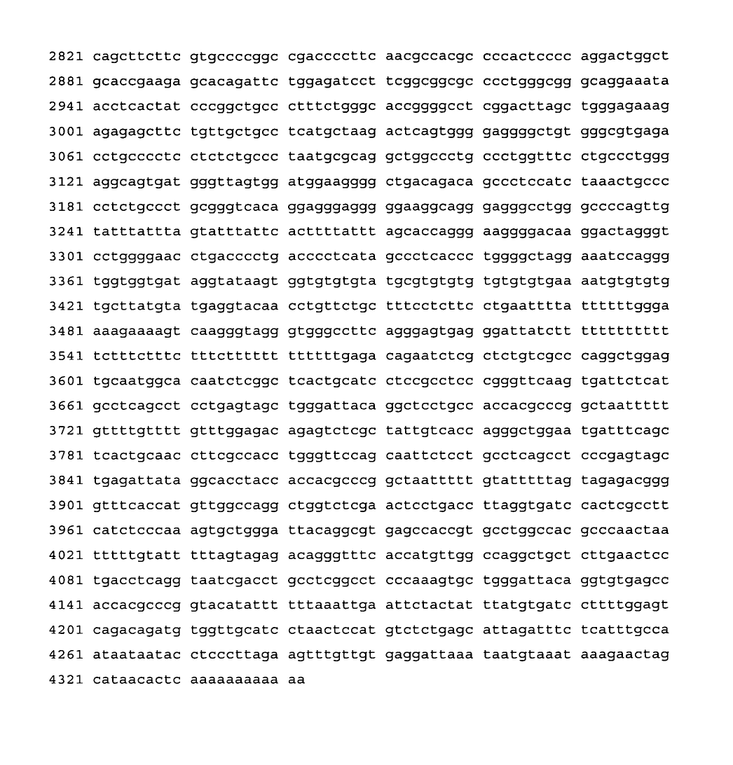

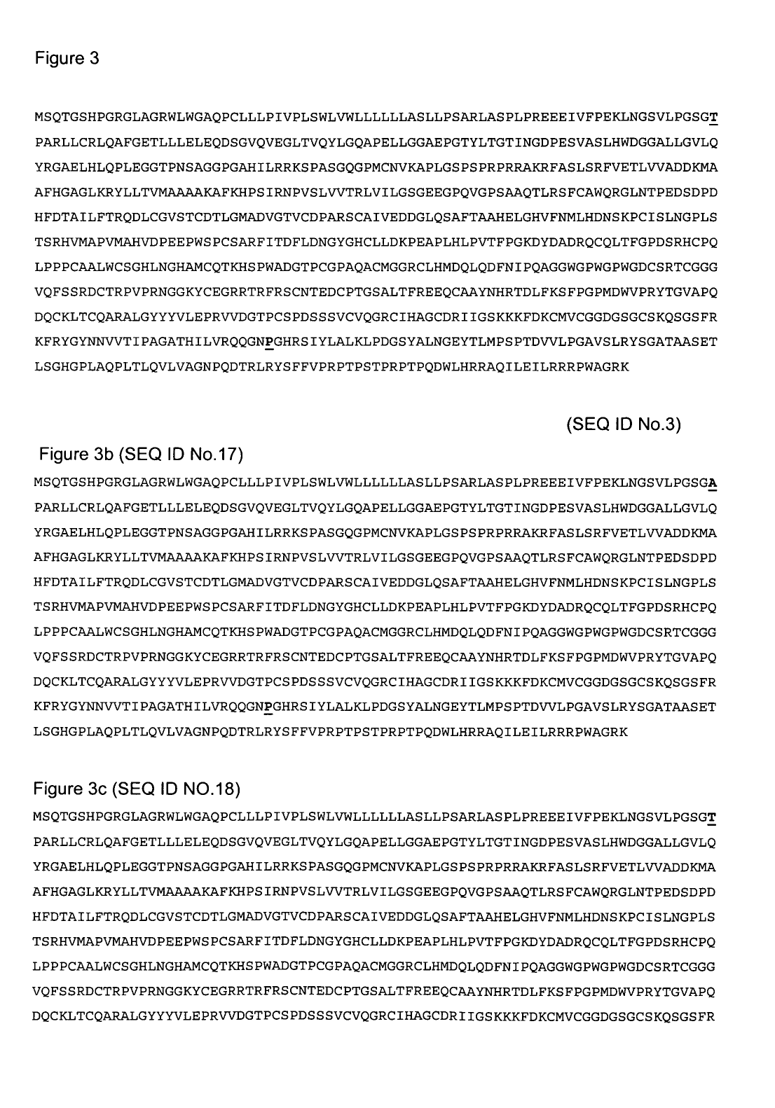

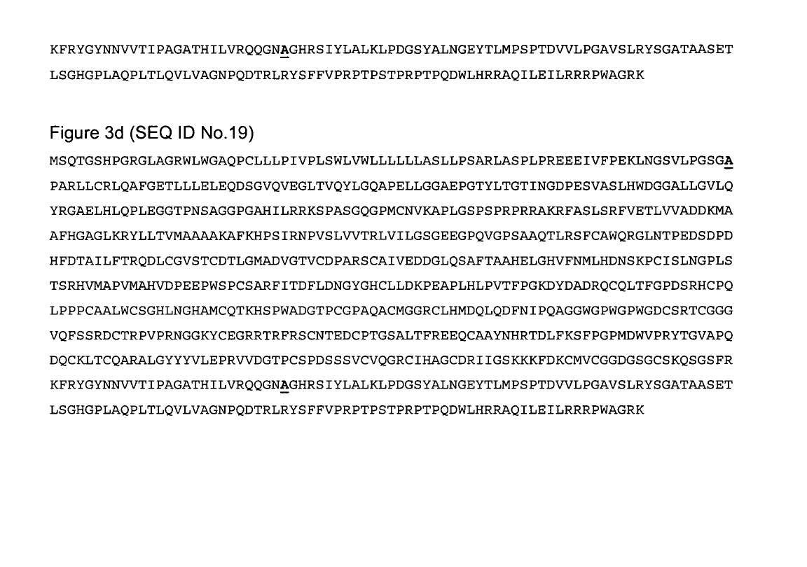

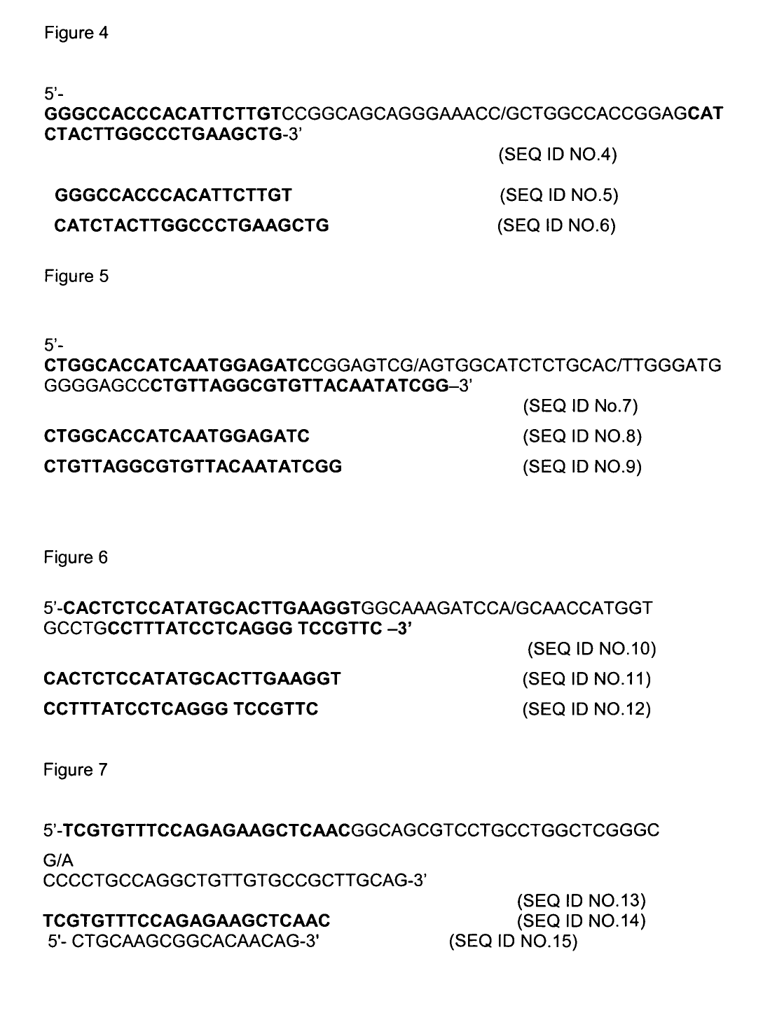

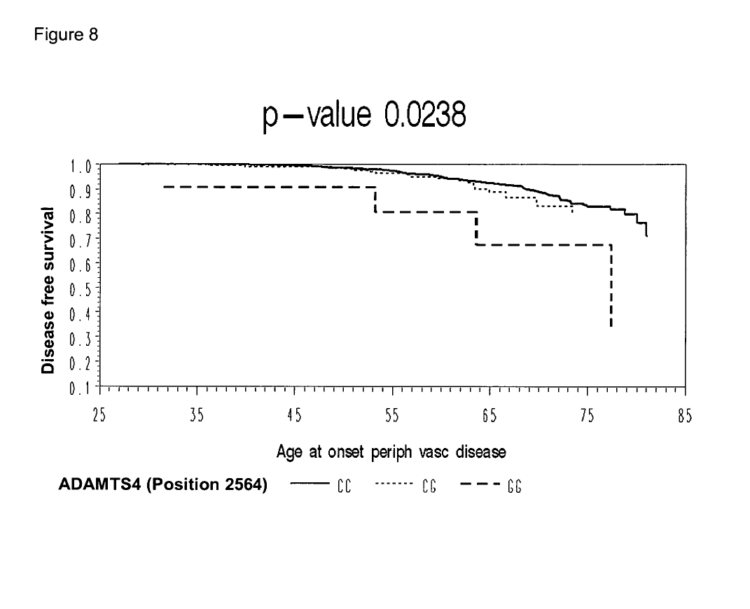

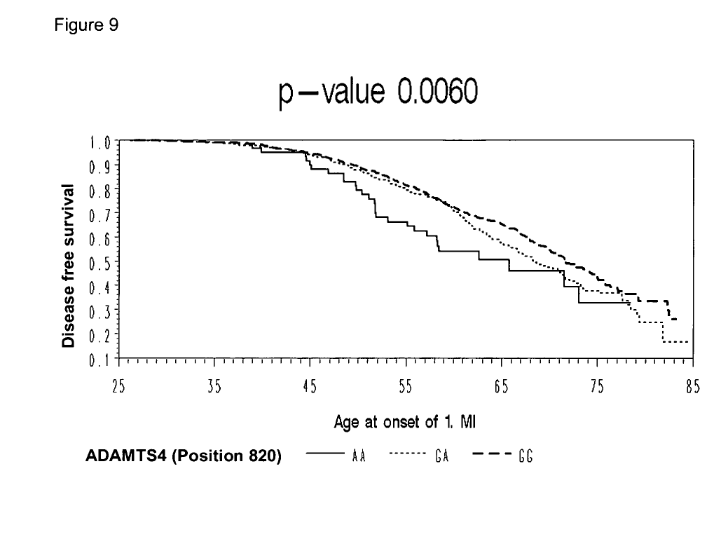

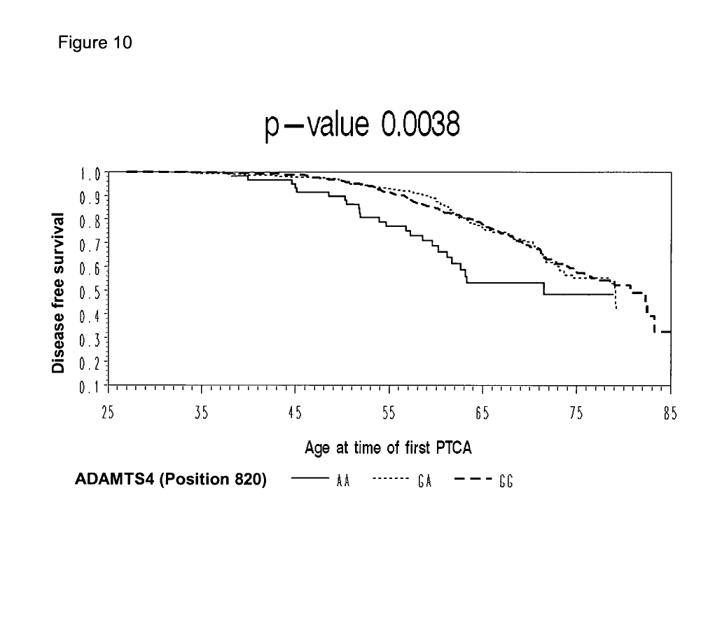

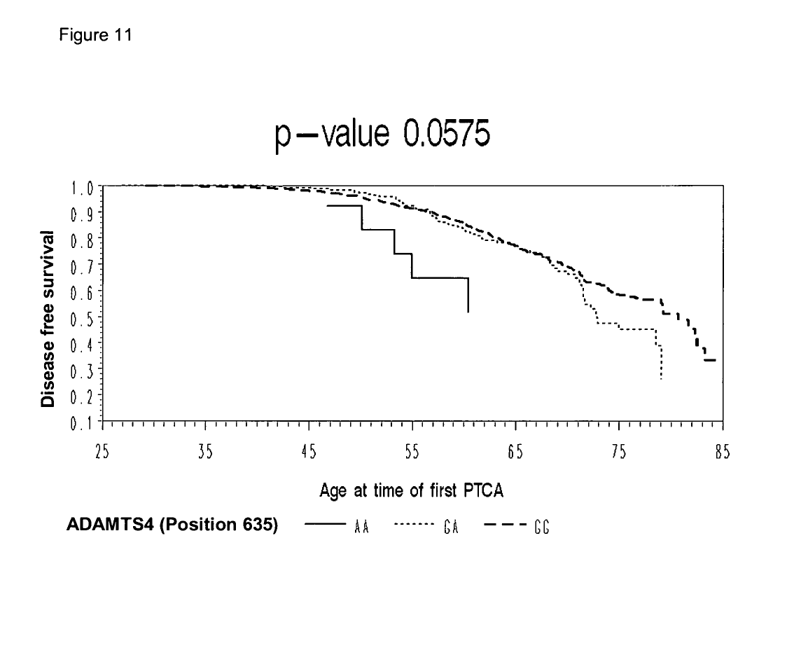

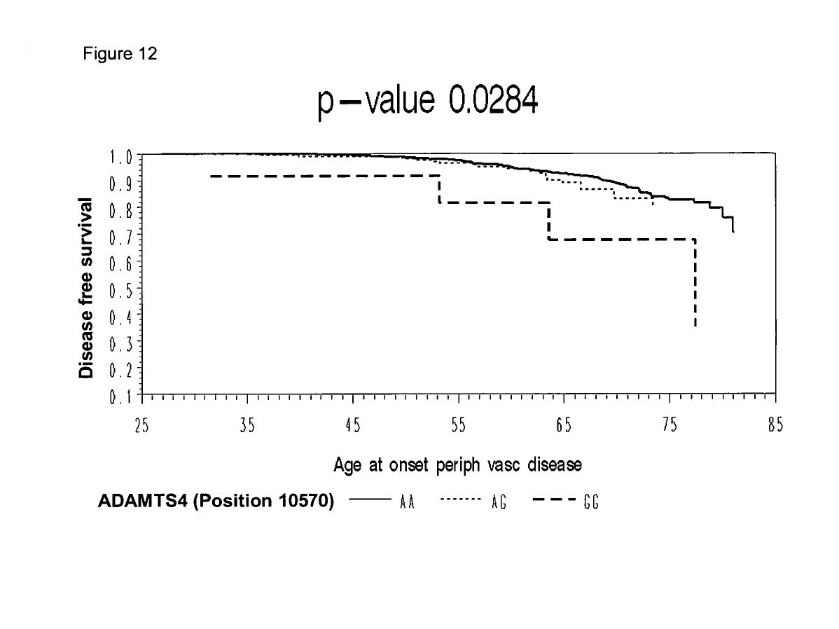

3a) Protein sequence of human ADAMTS4 (SEQ ID No.3) derived from the nucleic acid sequence with the NCBI Number NM_{—}005099.3 having a Threonine at position 77 and a Proline at position 720 (ADAMTS4 Thr77Pro720).

3b) Protein sequence of human ADAMTS4 derived from the nucleic acid sequence with the NCBI Number NM_{—}005099.3 having an Alanine at position 77 and a Proline at position 720 (ADAMTS4 Ala77Pro720).

3c) Protein sequence of human ADAMTS4 derived from the nucleic acid sequence with the NCBI Number NM_{—}005099.3 having Threoning at position 77 and an Alanine at position 720 (ADAMTS4 Thr77Ala720).

3d) Protein sequence of human ADAMTS4 derived from the nucleic acid sequence with the NCBI Number NM_{—}005099.3 having an Alanine at position 77 and at position 720 (ADAMTS4 Ala77Ala720).

FIG. 4: Sequence stretch (Positions 2529-2599, SEQ ID NO.4) of the human ADAMTS4 gene comprising the genetic variant C→G at Position 2564 of the reference sequence NM_{—}005099.2 (ADAMTS4-C2564G). For analysing this region in the gene, the primer set with the primers according to SEQ ID Nos. 5 and 6 was used.

FIG. 5: Sequence stretch (Positions 792-874; SEQ ID NO.7) of the human ADAMTS4 gene comprising the genetic variant G→A at position 820 of the reference sequence according to NM_{—}005099.2 (ADAMTS4-G820A) and/or the genetic variant C→T at position 835 of the reference sequence according to NM_{—}005099.2 (ADAMTS4-C835T). For analysing this region of the ADAMTS4 gene, the primer set comprising primers with the sequences according to SEQ ID NOs. 8 and 9 was used.

FIG. 6: Sequence stretch (Positions 10535-10607; SEQ ID NO. 10) of thr human ADAMTS4 gene comprising the genetic variant A→G at position 10570 of the reference sequence AY044847.1 (ADAMTS4-A10570G). For analysing this region in the human ADAMTS4 gene, the primer set with sequences according to SEQ ID NOs. 11 and 12 was used.

FIG. 7: Sequence stretch (Positions 588-664; SEQ ID NO. 13) of the human ADAMTS4 gene comprising the genetic variation G→A at position 635 of the reference sequence according to NM_{—}005099.2 (ADAMTS4-G635A). For analysing this region of the gene, the primer set with sequences according to SEQ ID NOs. 14 and 15 was used.

FIG. 8: Impact of the ADAMTS4 genotype at position 2564 of the reference sequence according to NM_{—}005099.2 (ADAMTS4-C2564G) on the onset (age) of peripheral vascular disorders (here peripheral vascular disease) in the patient cohort analysed. As can be gained from FIG. 8, the likeliness of experiencing peripheral vascular disorders earlier in life is higher in patients having the G2546G genotype than in patients with either the G2546C or C2546C (most frequent variant) genotype.

FIG. 9: Impact of the ADAMTS4 genotype at position 820 of the reference sequence according to NM_{—}005099.2 (ADAMTS4-A820G) on the onset (age) of a first heart attack (here MI=myocardial infarction) in the patient cohort analysed. As can be gained from FIG. 8, the likeliness of experiencing a first myocardial infarction earlier in life is higher in patients with the A820G genotype than in patients having G820G or A820A.

FIG. 10: Impact of the ADAMTS4 genotype at position 820 of the reference sequence according to NM_{—}005099.2 (ADAMTS4-A820G) on having (age) a first PTCA (percutaneous transluminal coronar angioplasty) in the patient cohort analysed. As can be gained from FIG. 10, the risk of having a PTCA undertaken earlier in life is higher in patients having the A820A genotype than in patients having G820G or A820G.

FIG. 11: Impact of the ADAMTS4 genotype at position 635 of the reference sequence according to NM_{—}005099.2 (ADAMTS-G635A) on having (age) a first PTCA (percutaneous transluminal coronar angioplasty) in the patient cohort analysed. As can be gained from FIG. 11, the risk of having a PTCA undertaken earlier in life is higher in patients having the A635A genotype than in patients having G635G or G635A.

FIG. 12: Impact of the ADAMTS4 genotype at position 10579 of the reference sequence according to AY044847.1 (ADAMTS4-A10570G) on the onset (age) of peripheral vascular disorders (here peripheral vascular disease) in the patient cohort analysed. As can be gained from FIG. 12, the risk of suffering from peripheral vascular diseases earlier in life is higher in patients having the G10579G genotype than in patients having the G10579A variant or the most frequent variant A10579A.

Table 1: Frequency and distribution of genetic variants at position 2564 of the human ADAMTS4 gene with reference sequence according to NM_{—}005099.2 (ADAMTS4-C2564G) in the patient cohort analysed. As can be gained from the table, the most frequent variant is C2546C.

Table 2: Frequency and distribution of genetic variants at position 835 of the human ADAMTS4 gene with reference sequence according to NM_{—}005099.2 (ADAMTS4-C835T) in the patient cohort analysed. As can be gained from the table, the most frequent variant is C835C; the variant T835T was not found in the patient cohort analysed.

Table 3: Frequency and distribution of genetic variants at position 820 of the human ADAMTS4 gene with reference sequence according to NM_{—}005099.2 (ADAMTS4-G820A) in the patient cohort analysed. As can be gained from the table, the most frequent variant is G820G.

Table 4: Frequency and distribution of genetic variants at position 10570 of the human ADAMTS4 gene with reference sequence according to AY044847.1 (ADAMTS4-A10570G) in the patient cohort analysed. As can be gained from the table, the most frequent variant is A10570A.

Table 5: Frequency and distribution of genetic variants at position 635 of the human ADAMTS4 gene with reference sequence according to NM_{—}005099.2 (ADAMTS4-G635A) in the patient cohort analysed. As can be gained from the table, the most frequent variant is G635G.

Table 6: Impact of the ADAMTS4 genotype at position 2564 of the reference sequence according to NM_{—}005099.2 (ADAMTS4-C2564G) on the onset of peripheral vascular disorders, high blood pressure (systolic blood pressure>=140 mm Hg and/or diastolic blood pressure>=90 mm Hg) and/or stroke/PRIND/TIA (PRIND=prolonged reversible ischemic neurologic deficite; TIA=transitoric ischemic attack) in the patient cohort analysed. As can be gained from the table, patients having the G2564G genotype have a significantly higher probability of suffering from peripheral vascular disorders, high blood pressure and/or stroke/PRIND/TIA than patients with one of the other two genotypes. Patients having the C2564G genotype have a little higher probability of suffering from peripheral vascular disease or from stroke/PRIND/TIA than patients with the C2564C genotype.

Table 7: Impact of the ADAMTS4 genotype at position 835 of the reference sequence according to NM_{—}005099.2 (ADAMTS4-C835T) on the onset of instable angina and/or early heart infarction (<=55 years of age with respect to men and <=60 years of age with respect to women) in the patient cohort analysed. As can be gained from the table, the probability of suffering from instable angina or early heart infarction is significantly higher in patients having the C835T genotype than in patients with the C385C genotype.

Table 8: Impact of the ADAMTS4 genotype at position 820 of the reference sequence according to NM_{—}005099.2 (ADAMTS4-G820A) the probability of having experienced an angio-coronary-plasty and/or the onset of heart infarction and/or early heart infarction (<=55 years of age with respect to men and <=60 years of age with respect to women) in the patient cohort analysed. As can be gained from the table, the probability of suffering from a heart infarction and/or an early heart infarction and/or having a coronary angioplasty undertaken is significantly higher in patients with the A820A genotype than in patients with the two other genotypes and somewhat higher in patients with the G820A genotype than in patients with the most frequent variant G820G.

Table 9: Impact of the ADAMTS4 genotype at position 10570 of the reference sequence according to AY044847.1 (ADAMTS4-A10570G) on the onset of peripheral vascular disorders in the patient cohort analysed. As can be gained from the table, the probability of suffering from peripheral vascular disease is significantly higher in patients having the G10570G genotype than in patients with one of the other two genotypes and somewhat higher in patients having the A10570G genotype versus patients with the most frequent variant A10570A.

Experiments:

1. SNP Detection by sequencing and analyses of sequencing results

1.a) amplification of DNA regions in the ADAMTS4 gene

Oligonucleotides (primers) for DNA amplification:

For detection of the nucleotide present at position 635 in the ADAMTS4 gene the following primers were used:

Primer 1: 5′-TCGTGTTTCCAGAGAAGCTCAAC-3′
(SEQ ID No. 14, positions 588-610 of the reference
sequence NM_005099.2)
Primer 2: 5′-CTGCAAGCGGCACAACAG-3′
(SEQ ID No. 15, positions 664-647 of the reference
sequence NM_005099.2)

For detection of the nucleotide present at position 820 in the ADAMTS4 gene the following primers were used:

Primer 1: 5′-CTGGCACCATCAATGGAGATC-3′
(SEQ ID No. 8, positions 792-812 of the reference
sequence NM_005099.2)
Primer 2: 5′-CCGATATTGTAACACGCCTAACAG-3′
(SEQ ID No. 9, positions 874-851 of the reference
sequence NM_005099.2)

For detection of the nucleotide present at position 835 in the ADAMTS4 gene the following primers were used:

Primer 1: 5′-CTGGCACCATCAATGGAGATC-3′
(SEQ ID No. 8, positions 792-812 of the reference
sequence NM_005099.2)
Primer 2: 5′-CCGATATTGTAACACGCCTAACAG-3′
(SEQ ID No. 9, positions 874-851 of the reference
sequence NM_005099.2)

For detection of the nucleotide present at position 2564 in the ADAMTS4 gene the following primers were used:

Primer 1: 5′-GGGCCACCCACATTCTTGT-3′
(SEQ ID No. 5, positions 2529-2547 of the
reference sequence NM_005099.2)
Primer 2: 5′-CAGCTTCAGGGCCAAGTAGATG-3′
(SEQ ID NO. 6, positons 2599-2578 of the reference
sequence NM_005099.2)

For detection of the nucleotide present at position 10570 in the ADAMTS4 gene the following primers were used:

Primer 1: 5′-CACTCTCCATATGCACTTGAAGGT-3′
(SEQ ID No. 11, positions 10535-10558 of the
reference sequence AY044847.1)
Primer 2: 5′-GAACGGACCCTGAGGATAAAGG-3′
(SEQ ID No. 12, positions 10607-10586 of the
reference sequence AY044847.1)

PCR protocol for amplification:

Reagents used are from Applied Biosystems (Foster City, USA):

20 ng of genomic DNA; 1 unit of TaqGold DNA polymerase; 1×Taq polymerase buffer; 500 μM dNTPs; 2.5 mM MgCl₂; 200 nM of each amplification primer pair (sequences under 1.A); H₂O to 5 μl.

PCR amplification program for genotyping:

95° C. for 10 min×1 cycle

95° C. for 30 s

70° C. for 30 s×2 cycles;

95° C. for 30 s

65° C. for 30 s×2 cycles;

95° C. for 30 s

60° C. for 30 s×2 cycles;

95° C. for 30 s

56° C. for 30 s

72° C. for 30 s×40 cycles;

72° C. for 10 min

4° C. for 30 s×1 cycle;

Identification of SNPs

Protocol for minisequencing and detection of SNPs

All reagents are from Applied Biosystems (Foster City, USA). 2 μl of purified PCR product, 1.5 μl of BigDye terminator kit, 200 nM sequencing primer (for sequences, see under 1.A), H₂O to 10 μl.

Amplification program for sequencing:

96° C. for 2 min×1 cycle;

96° C. for 10 s

55° C. for 10 s

65° C. for 4 min×30 cycles;

72° C. for 7 min

5 4° C. for 30 s×1 cycle;

Analysis of sequencing products:

The sequences were firstly analyzed using the “Sequenz Analyse Software” (Applied Biosystems, Foster City, USA) to obtain the raw data and then methoded with Phred, Phrap, Polyphred and Consed. Phred, Phrap, Polyphred and Consed are software written by Phil Green at Washington University (http://www.genome.washington.edu).

### EXAMPLE 2

### Statistical Analysis of the Identified SNPs

The newly identified ADAMTS4 polymorphisms of the reference sequences NM_{—}005099 and AY044847.1 were analysed for association with clinical parameters in about 1400 patients. The frequency and distribution of the different identified polymorphisms can be gained from tables 1 to 5. Associations of the identified variants G/A at positions 635, 820, 835, 2564 of the reference sequence NM_{—}005099 and at position 10570 with respect to the reference sequence AY044847.1 with clinical endpoints in the patient group analyzed can be found in FIGS. 8 to 12 and tables 6 to 10. All statistical analyses were carried out with SAS version 8.2 (SAS Institute GmbH, Heidelberg, Germany).

The p value is a parameter relating to the statistical significance of the associations observed, RR (risk ratio) is a parameter relating to the increased risk of the occurrence of the clinical end point indicated in patients having a certain ADAMTS4 polymorphism. RR was calculated with adjustment of the patient groups with respect to age, sex, smoker, blood pressure and cholesterol level.

The patient group analyzed revealed a statistically significant age-dependent association of the early onset of peripheral vascular disorders in patients with the genotype ADAMTS4-G2564G in comparison to ADAMTS4-G2564C and ADAMTS4-C2564C (figure8). Moreover, the risk for suffering from peripheral vascular disorders, high blood pressure and stroke/PRIND/TIA is significantly increased in carriers of the ADAMTS-G2564G genotype in comparison to ADAMTS4-G2564C and ADAMTS4-C2564C (figure8).

In carriers of the genotype ADAMTS4-A820A a first heart-infarction and a first PTCA occurred significantly earlier in life than in carriers of the ADAMTS4-G820A or ADAMTS4-G820G genotype (figure9), moreover, these patients had a statistically significant higher risk for the necessity of undergoing a PTCA as sign of coronary degenerations/disorders and the occurrence of premature heart infractions and heart infarctions in comparison to carriers of the other two genotypes.

The undergoing of a PTCA occurred significantly earlier in life in patients with the ADAMTS4-A635A genotype in comparison to the ADAMTS4-G635A or ADAMTS4-G635G genotype (FIG. 10).

Peripheral vascular disorders occurred significantly earlier in life in carriers of the ADAMTS4-G10570G genotype in comparison to the ADAMTS4-G10570A and ADAMTS4-A10570A genotype (FIG. 12).

Carriers of the ADAMTS4-C835T genotype had a statistically significant higher risk to suffer from instable angina and premature heart infarction in comparison to carriers of the ADAMTS-C835C genotype (FIG. 13). Carriers of the ADAMTS4-T835T genotype could not be detected in the analyzed patient cohort, which may—perhaps—be attributable to a particularly severe phenotypic effect of this genotype, considering the relatively strong impact of the ADAMTS4-C835T genotype.

As one outcome of said study it can be generally stated that:
An Adenosine at position 635 of the genomic ADAMTS4 sequence on one or both alleles of the ADAMTS4 gene indicates an increased risk of suffering from or developing a disorder necessitating the undertaking of a coronary angioplasty, especially earlier in life than compared to individuals not having an Adenosine, and especially compared to individuals having a Guanosine at position 635 of the genomic ADAMTS4 sequence.
An Adenosine at position 820 of the genomic ADAMTS4 sequence on one or both alleles of the ADAMTS4 gene indicates an increased risk of experiencing a heart infarction and/or disorders necessitating the undertaking of a coronar angioplasty and especially suffering from one or more of said disorders earlier in life than compared to individuals not having an Adenosine, and especially having a Guanosine, at position 820 of the ADAMTS4 gene.
A Thymidine at position 835 of the genomic ADAMTS4 sequence on one allele of the ADAMTS4 gene indicates an increased risk of suffering from instable angina and/or heart infarction, and especially experiencing one or both of said disorders earlier in life than individuals not having a Thymidine, and especially having a Cytidine, at position 835 of the ADAMTS4 gene.
A Guanosine at position 2564 of the genomic ADAMTS4 sequence on one or both alleles of the ADAMTS4 gene indicates an increased risk of suffering from peripheral vascular disease and/or heart infarction and/or high blood pressure and/or stroke/PRIND/TIA, and especially suffering from one or more of said diseases earlier in life than individuals not having a Guanosine, and especially having a Cytidine, at position 2564 of the ADAMTS4 gene.
A Guanosine at position 10570 of the genomic ADAMTS4 sequence on one or both alleles of the ADAMTS4 gene indicates an increased risk of suffering from peripheral vascular disease, and especially suffering from peripheral vascular disease earlier in life than individuals not having a Guanosine, and especially having a Adenosine, at position 10570 of the ADAMTS4 gene.
A Threonine at position 77 of the polypeptide chain of the ADAMTS4 protein indicates an increased risk of suffering from a disease necessitating the undertaking of a coronar angioplasty, and especially of suffering from said disease earlier in life than individuals not having a Threonine, and especially having an Alanine, at position 77 of polypeptide chain of the ADAMTS4 protein.
An Alanine at position 720 of the polypeptide chain of the ADAMTS4 protein indicates an increased of suffering from peripheral vascular disease and/or heart infarction and/high blood pressure and/or stroke, especially to suffer from one or more of these disorders earlier in life than individuals not having an Alanine, and especially having a Proline, at position 720 of the polypeptide chain of the ADAMTS4 protein.

The associations between clinical endpoints and genetic variations of the ADAMTS4 nucleic acid and/or protein are a clear hint to the impact of genetic variants of ADAMTS4 on the onset of cardiovascular and peripheral vascular disorders such as coronary heart diseases, malfunctions in the hemostasis of the blood vessels and a tendency to occlusive disorders of the blood vessels. The statistically significant correlation between variants of ADAMTS4 and the onset of said clinical endpoints demonstrated here for the first time thus provides a meaningful basis for present invention.

TABLE 1
ADAMTS4-C2564G

| | | |
|---|---|---|
| ADAMTS4- | ADAMTS4- | ADAMTS4- |
| C2564C | C2564G | G2564G |

| n | % | n | % | n | % |
|---|---|---|---|---|---|
| 1208 | 85.01 | 202 | 14.22 | 11 | 0.77 |

TABLE 2
ADAMTS4-C835T

| | |
|---|---|
| ADAMTS4- | ADAMTS4- |
| C835C | C835T |

| n | % | n | % |
|---|---|---|---|
| 1354 | 94.95 | 72 | 5.05 |

TABLE 3
ADAMTS4-G820A

| | | |
|---|---|---|
| ADAMTS4- | ADAMTS4- | ADAMTS4- |
| G820G | G820A | A820A |

| n | % | n | % | n | % |
|---|---|---|---|---|---|
| 901 | 63.67 | 445 | 32.16 | 59 | 4.17 |

TABLE 4
ADAMTS4-A10570G

| | | |
|---|---|---|
| ADAMTS4- | ADAMTS4- | ADAMTS4- |
| A10570A | A10570G | G10570G |

| n | % | n | % | n | % |
|---|---|---|---|---|---|
| 1169 | 84.82 | 202 | 14.33 | 12 | 0.85 |

TABLE 5
ADAMTS4-G635A

| | | |
|---|---|---|
| ADAMTS4- | ADAMTS4- | ADAMTS4- |
| G635G | G635A | A635A |

| n | % | n | % | n | % |
|---|---|---|---|---|---|
| 1148 | 81.19 | 253 | 17.89 | 13 | 0.92 |

TABLE 6

| | ADAMTS4 | | | | | | |
|---|---|---|---|---|---|---|---|
| | ADAMTS4- | | ADAMTS4- | | ADAMTS4- | | |
| | C2564C | | C2564G | | G2564G | | |
| | n | % | n | % | n | % | p-Wert |
| Periphere | 94 | 7.78 | 21 | 10.40 | 4 | 36.36 | 0.0016 |
| vaskuläre | | | | | | | |
| Krankheit | | | | | | | |
| Bluthochdruck | 637 | 52.73 | 105 | 51.98 | 10 | 90.91 | 0.0395 |
| Schlaganfall/ | 85 | 7.04 | 17 | 8.42 | 3 | 27.27 | 0.032 |
| PRIND/TIA | | | | | | | |

TABLE 7

| | ADAMTS4 | | | | |
|---|---|---|---|---|---|
| | ADAMTS4- | | ADAMTS4- | | |
| | C835C | | C835T | | |
| | n | % | n | % | p-Wert |
| Instable Angina | 413 | 30.5 | 31 | 43.03 | 0.025 |
| frühzeitiger Herzinfarkt | 248 | 18.31 | 20 | 27.78 | 0.0452 |

TABLE 8

| | ADAMTS4 | | | | | | |
|---|---|---|---|---|---|---|---|
| | ADAMTS4- | | ADAMTS4- | | ADAMTS4- | | |
| | G820G | | G820A | | A820A | | |
| | n | % | n | % | n | % | p-Wert |
| Koronarangioplastie | 224 | 25.11 | 111 | 25.28 | 25 | 43.86 | 0.0069 |
| Herzinfarkt | 348 | 38.62 | 206 | 45.27 | 30 | 50.85 | 0.0198 |
| frühzeitiger | 162 | 17.98 | 90 | 19.78 | 19 | 32.2 | 0.0247 |
| Herzinfarkt | | | | | | | |

TABLE 9

| | ADAMTS4 | | | | | | |
|---|---|---|---|---|---|---|---|
| | ADAMTS4- | | ADAMTS4- | | ADAMTS4- | | |
| | A10570A | | A10570G | | G10570G | | |
| | n | % | n | % | n | % | p-Wert |
| Periphere | 93 | 7.78 | 21 | 10.40 | 4 | 33.33 | 0.0034 |
| vaskuläre | | | | | | | |
| Krankheit | | | | | | | |

1-48. (canceled)

49. A nucleic acid probe with the nucleic acid sequence according to SEQ ID No. 4, 7, 10 or 13.

50. A method for identifying cardiovascular and peripheral vascular disorders or an increased risk for developing cardiovascular and peripheral vascular disorders in an individual, which comprises examining a sample taken from an individual for the type of nucleotide which is present on one or more of the positions 635, 820, 835, 2564 and 10570 on one or both alleles of the ADAMTS4 gene, the type of nucleotide present at said one or more positions being indicative of the risk of said individual to suffer from or develop cardiovascular and peripheral vascular disorders.

51. A method for identifying cardiovascular and peripheral vascular disorders or an increased risk for developing cardiovascular and peripheral vascular disorders in an individual, which comprises examining a sample taken from an individual for the type of amino acid present at one or both of the positions 77 and 720 of the polypeptide chain of ADAMTS4 protein, the type of amino acid present at said one or more positions being indicative of the risk of said individual to suffer from or develop cardiovascular and peripheral vascular disorders.

52. A method for identifying cardiovascular and peripheral vascular disorders or an increased risk for developing cardiovascular and peripheral vascular disorders in an individual, which comprises examining a sample taken from an individual and determining the amount of ADAMTS4 mRNA present in said sample is different from that of one or more reference samples the presence of a different amount of ADAMTS4 mRNA indicating an increased risk to suffer from or develop cardiovascular and peripheral vascular disorders.

53. A method for identifying cardiovascular and peripheral vascular disorders or an increased risk for developing cardiovascular and peripheral vascular disorders in an individual, which comprises examining a sample taken from an individual and determining the amount of ADAMTS4 protein present in said sample is different from that of one or more reference samples the presence of a different amount of ADAMTS4 protein indicating an increased risk to suffer from or develop cardiovascular and peripheral vascular disorders.

54. A method of determining the risk of suffering from cardiovascular and peripheral vascular disorders comprising analysing an isolated sample of an individual for the type of nucleotide which is present on one or more of the positions 635, 820, 835, 2564 and 10570 on one or both alleles of the ADAMTS4 gene and calculating the estimated risk on basis of the age and the type of nucleotide present at one or more of the positions 635, 820, 835, 2564 and 10570 in the ADAMTS4 gene.

55. A method of determining the risk of suffering from cardiovascular and peripheral vascular disorders comprising analysing an isolated sample of an individual for the presence of one or more ADAMST4 protein polymorphisms and calculating the estimated risk on basis of the age and the type of amino acid present at position 77 or 720 of the ADAMTS4 protein.

56. A method for selecting a pharmaceutical or adapting the dosage of a pharmaceutical for the prevention and/or treatment of cardiovascular and peripheral vascular disorders in an individual, which comprises examining a taken sample of the individual for the type of the nucleotide which is present at one or more of the positions 635, 820, 835, 2564 and 10570 on either or both alleles of the ADAMTS4 gene, wherein said pharmaceutical being selected or said dosage being adapted is dependent of the type of nucleotide present at one or more of said positions.

57. A method for selecting a pharmaceutical or adapting the dosage of a pharmaceutical for the prevention and/or treatment of cardiovascular and peripheral vascular disorders in an individual, which comprises examining a taken sample of the individual for the type of amino acid present at one or both of the positions 77 and 720 in the amino acid chain of the ADAMTS4 protein, wherein said pharmaceutical being selected or said dosage being adapted is dependent of the type of amino acid present at one or both of said positions.

58. A method for selecting a pharmaceutical or adapting the dosage of a pharmaceutical for the prevention and/or treatment of cardiovascular and peripheral vascular disorders in an individual, which comprises examining a taken sample of the individual as to whether the amount of ADAMTS4 mRNA present in said sample is different from that of one or more taken reference samples said pharmaceutical being selected or said dosage being adapted depending on whether the amount of ADAMTS4 mRNA in the taken sample of the individual is different from that of the reference sample or reference samples.

59. A method for selecting a pharmaceutical or adapting the dosage of a pharmaceutical for the prevention and/or treatment of cardiovascular and peripheral vascular disorders in an individual, which comprises examining a taken sample of the individual as to whether the amount of ADAMTS4 protein present in said sample is different from that of one or more taken reference samples said pharmaceutical being selected or said dosage being adapted depending on whether the amount of ADAMTS4 protein in the taken sample of the individual is different from that of the reference sample or reference samples.

60. A method for identifying substances active in preventing or treating cardiovascular and peripheral vascular disorders comprising:
a) contacting a ADAMTS4 protein or functional fragment or derivative thereof with a test substance; and
b) determining whether the test substance modulates the activity of the ADAMTS4 protein or functional fragment or derivative thereof.

61. A method for identifying substances active in preventing or treating cardiovascular and peripheral vascular disorders comprising:
a) contacting a cell, which has a detectable amount or activity of ADAMTS4 or of a functional fragment or derivative thereof, with a test substance;
b) determining whether the test substance is able to modulate the amount or activity of ADAMTS4 or the functional fragment or derivative thereof present in the cell.

62. A test kit for diagnosing cardiovascular and peripheral vascular disorders or a predisposition for cardiovascular and peripheral vascular disorders, the test kit comprising at least one means for the detection of ADAMTS4 gene, mRNA or protein in one or more biological samples.

63. The method as claimed in claim 50, wherein one or both alleles of the ADAMTS4 gene have one or more genomic variants, the genomic variant selected from the group consisting of an adenosine at position 635 of the genomic ADAMTS4 sequence, an adenosine at position 820 of the genomic ADAMTS4 sequence, a thymidine at position 835 of the genomic ADAMTS4 sequence, a guanosine at position 2564 of the genomic ADAMTS4 sequence, and a guanosine at position 10570 of the genomic ADAMTS4 sequence.

64. The method as claimed in claim 51, wherein the ADAMTS4 protein has one or both protein variants, the protein variants selected from the group consisting of a threonine at position 77 of the polypeptide chain and an alanine at position 720 of the polypeptide chain.

65. The method as claimed in claim 50, wherein one or both alleles of the ADAMTS4 gene have one or more of the following genomic variants, the genomic variants selected from the group consisting of a nucleotide other than adenosine, and preferably a guanosine, at position 635 of the genomic ADAMTS4 sequence on one or both alleles of the ADAMTS4 gene; a nucleotide other than adenosine, and preferably a guanosine, at position 820 of the genomic ADAMTS4 sequence on one or both alleles of the ADAMTS4 gene; a nucleotide other than thymidine, and preferably a cytidine, at position 835 of the genomic ADAMTS4 sequence on both alleles of the ADAMTS4 gene; a nucleotide other than guanosine, and preferably a cytidine, at position 2564 of the genomic ADAMTS4 sequence on one or both alleles of the ADAMTS4 gene; and a nucleotide other than guanosine, and preferably a adenosine, at position 10570 of the genomic ADAMTS4 sequence on one or both alleles of the ADAMTS4 gene.

66. The method as claimed in claim 51, wherein the ADAMTS4 protein has one or both protein variants, the protein variants selected from the group consisting of an amino acid other than threonine, and preferably an alanine, at position 77 of the polypeptide chain of the ADAMTS4 protein; and an amino acid other than alanine, and preferably a proline, at position 720 of the polypeptide chain of the ADAMTS4 protein.

67. The method of claim 50, wherein the cardiovascular disorder is selected from the group consisting of angina pectoris, instable angina pectoris, heart infarction, early heart infarction, peripheral vascular disorder, high blood pressure, stroke, PRIND, TIA and a disorder necessitating the undertaking of a coronary angioplasty.

68. The method of claim 50, comprising analyzing a taken sample of an individual, wherein the individual whose taken sample is examined has a glucose metabolism disorder, preferably suffering from diabetes and particularly preferably from type 1 diabetes.

69. The method of claim 50, wherein the individual whose taken sample is examined suffers from hypertension and/or has already suffered a myocardial infarction.

70. The method of claim 50, wherein the sample is a mammalian and preferably a human sample.

71. The method of claim 50, wherein the sample is selected from the group consisting of a histological sample, a biopsy sample, a cell extract, one or more cells and taken body fluid.

72. The method of claim 50, wherein the type of nucleotide at one or more positions selected from the group consisting of 635, 820, 835, 2564 and 10570 in the ADAMTS4 gene is determined by means of one or more suitable primers or probes.

73. The method of claim 50, wherein the type of nucleotide is identified by means selected from the group consisting of PCR, Southern Blot, Array-hybridisation and Chip-hybridisation.

74. The test kit according to claim 62, wherein the means for the detection is a means for the detection of ADAMTS4 DNA.

75. The test kit according to claim 62, wherein the means is selected from the group consisting of a primer or set of primers, a suitable probe and a sequence-specific anti-DNA antibody.

76. The method of claim 52, wherein the change in the amount of mRNA is analyzed, preferably using one or more suitable primers for the amplification of ADAMTS4 cDNA or one or more probes suitable for hybridization with ADAMTS4 cDNA or mRNA at standard conditions.

77. The method according to claim 76, wherein the amount of mRNA is analysed by means selected from the group consisting of PCR, Northern Blot, Array-hybridisation and Chip-hybridisation.

78. The test kit as claimed in claim 62, wherein the means for detection is a means for detecting ADAMTS4 mRNA and/or protein present in a biological sample.

79. The test kit as claimed in claim 62, wherein the means for detecting ADAMTS4 protein is an antibody.

80. The method according to claim 51, wherein the protein is detected with the aid of at least one antibody.

81. The method of claim 51, wherein the protein is detected by a method selected from the group consisting of ELISA, Western blot and protein chip.

82. The method of claim 80 or 81, wherein detection is carried out by means of immunohistochemical or immunoradiochemical detection methods.

83. The method of claim 50, wherein the genomic ADAMTS4 nucleic acid sequence is the sequence as defined in SEQ ID No. 1 or 16, optionally with a deviation with respect to one or more of the nucleotides at positions 635, 820, 835, 2564 and 10570.

84. The method of claim 83, wherein the sequence has one or more single nucleotide polymorphisms (SNPs), the SNPs selected from the group consisting of an adenosine at position 635 of the genomic ADAMTS4 sequence, an adenosine at position 820 of the genomic ADAMTS4 sequence, a thymidine at position 835 of the genomic ADAMTS4 sequence, a guanosine at position 2564 of the genomic ADAMTS4 sequence and a guanosine at position 10570 of the genomic ADAMTS4 sequence.

85. The method of claim 50, wherein the type of nucleotide present is detected by a primer set containing one or more primers to the sequence selected from the group consisting of SEQ ID No.5, 6, 8, 9, 11, 12, 14 and 15 or a probe as defined by the sequence selected from the group consisting of SEQ ID No. 4, 7, 10 and 13.

86. An isolated nucleic acid, having a genomic ADAMTS4 sequence according to SEQ ID No. 1 or 16 and comprising one or more single nucleotide polymorphisms (SNPs), the SNPs selected from the group consisting of an adenosine at position 635 of the genomic ADAMTS4 sequence, an adenosine at position 820 of the genomic ADAMTS4 sequence, a thymidine at position 835 of the genomic ADAMTS4 sequence, a guanosine at position 2564 of the genomic ADAMTS4 sequence and a guanosine at position 10570 of the genomic ADAMTS4 sequence.

87. An isolated ADAMTS4 protein having one or both protein polymorphisms selected from the group consisting of a threonine at position 77 of the polypeptide chain of the ADAMTS4 protein and an alanine at position 720 of the polypeptide chain of the ADAMTS4 protein.

88. The isolated ADAMTS4 protein according to claim 88 having the sequence selected from the group consisting of SEQ ID No. 3, 17, 18 and 19.

89. A nucleic acid primer with a nucleic acid sequence selected from the group consisting of SEQ ID No. 5, 6, 8, 9, 11, 12, 14 and 15.


SEQUENCE LISTING (19 sequences)

SEQ ID NO: 1 - DNA, 4307 bp, Homo sapiens
cacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcacccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggacctgtgtggagtctccacttgcgacacgctgggtatggctgatgtgggcaccgtctgtgacccggctcggagctgtgccattgtggaggatgatgggctccagtcagccttcactgctgctcatgaactgggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatgggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccgggcactgggctactactatgtgctggagccacgggtggtagatgggaccccctgttccccggacagctcctcggtctgtgtccagggccgatgcatccatgctggctgtgatcgcatcattggctccaagaagaagtttgacaagtgcatggtgtgcggaggggacggttctggttgcagcaagcagtcaggctccttcaggaaattcaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccagctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataac

SEQ ID NO: 2 - DNA, 10766 bp, Homo sapiens
accctaatttccctatatgcacaatgctgtctccaccctctccctgccccagggagaattaaaaagaaaagatgactagatattccaggaaccactgggttctcagagcaaggtggggtggatggtgggagccaggtggggattctcccagattgatactgggtgaatctgggttcctgagagcaagtcttgcctatgctgggggctggctgacttgaggctgggggagggtttagggcagttgggagtgggtaggagcagggccaaaagcctgggggaagctactgggagctgggccagggaaatggggagtcaggaagtggggagggggaaccctggggggaaatggaggcggaatggctgttctgggctttggagggggtgggtagtggtaactcaggaagggggatcctgagggagagaagggacgttagaaaagaggaggtgccaccctggatccgccttctataaaaggaaaagtcgttaacccctcctgccttgtcatctgccgcctctgttatgttcattccaagcaggatcatcctacctttgggcagtcaactccctgatcactgtctccttgcctcccccaatgttctgccttttttactcttcccagctgctcagttctatcctgagccatgtcaagctacctcttttatttgttcttccctcttgatgcctccttacctgttccctaccctcttttctcaggcagctcactcagtcccctcagccctggaaaccagccactagggccaaagggcagcatgagggagccttgagaaaagagaagccatggtaggttagactataagagcaggaattctcccaggaccgtgatcctatctgtgcatgccggccaggccctttccctcactctctgcctctcctggggctctgtcccaccaaaaagggaaagagacagctgagggctgattgtggggtttgggaaaaggctatgtcatcagctggcccagtgcctattatccattcggctgctagagattcccctcccctgggcaagtcccatttttttgggaagcgatgatacacccatctgagtcccaccgacagagctcagctgagtggcttagagatcagccaatcaatcgcagaggctcaccatgcttaaaagagctggcgcggagagaggctggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcgcccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaaggtaggcatctctggagtctgtgtcctgcggtgtcctcccgccatgtctgtcctactgacccctattcaccctctctccacccacttacacttctggccgttctgcttccctcccttctgcctgcctttcctttctccatgagcctcctttcttggtaaggctcaggcctctccccttcacccttctcctagccagacccaccgagggttctcagaggtgggtcacagactctgggctgggaataactgctccctctccccaccccaccccttcctggccatggcatctcctactgacacagtccccagcagtaaaggtctcctaacagccccttggccttggctcttttctctgctccaaatttgattttcatcccccactcccagctataggagtcaggtttgaggccctagggcttggtgtctggcttctgatgctgcctttaaaatcactgtggcaaactgcaaactagggcaggagtggcaagagagggagggtatcccatacctccttctggtcaatccccttcacacactgtggccggactgcgaggctgcctgaggctccatcctttcctccctcccactcctggtctcccccttcttcccttccctcctccctcctccctctctagctttaatccagaccagacgctccctccacaaccgccacgctgagctctggccctggccctagctgctgactgaatcgttcctccaaccacacagggcagacacgttgcagcccagccctctagccctggtcccataggccccagccacactcaggcctggagaaccccaaagtccctcctgacggcagtccaggctgctttcacctctggggctcccgcagccctgggatgtggcgtccccttctcctccccttcttagaaccagctgctgactcctcagagttaccacaaaagccctgagatttggcgttgggggaagggcccaaccaagacccgccaggagtcaggaatcccgtggggtgtgtggggcccagctgactatgctgagttccagacccaggaagtgagggattggagaatcgaccgacccatgggtctgagaggctatttcaggatcttcctctccctttttctctacagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggtgaggccccagtcagcaccgcaggccatgcactgcacaattctaagaagcgccactacatagactatgatgcaaatgatgttcctggagttgtgtgacatggtgccttgccccagccacactgccatgtgtggctacctaggccctgacagtgctgggtcctcagtgcccaggtccccgggaatctggatctcaacccaatggacccaggtgtcttggctgtctatgcaccttctatgatccctctccacaggacctgtgtggagtctccacttgcgacacgctgggtatggctgatgtgggcaccgtctgtgacccggctcggagctgtgccattgtggaggatgatgggctccagtcagccttcactgctgctcatgaactgggtaaagtaggggtggatgagaaaggtattagggaggagaaggtgggggagggggtagcaagttcaccacagtgttaatgggggtcccaaggtattcttcccccaggcctaggtatagggctattactcctctctgctccaggtgtagacatacatttacatttttctattaaataaattagttggtcgaaactactgtgaactcctctgtgtgtgtgtgtgtgtgtgtgtgtgtgtctgtgtgtgtgtatttggagggggagtcagagacaatgggtggggtcacagttgggaatgttggctataaggaaaagtgggtagggagggaccctgagtatattttggggaagaggagacagagaaagagacggggctacatactgggagccctgagagtaccataaaggaaaggactgggggcagggtgaggtgggtaatggatctgatcatccacagctgcagccagatttattatggagtccctcagggcatggggaagggaccctcagatcctatcagggcctttgctcccacaggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatggtaagcagatggcacttacctttccctgggtagcactcaattcctgtcctccagggcactctcacttccgctgcattctgcatggaccaaattgcctcccccaagtgtgcttcctctgcagtgtgggaaggggtgagaaatgctgctgtctttgtgtagttctcacttcctcccagaataaatagcatagctagaggaggcgaattttggctcagtagaattagaactgactttctgagggccagaactgtccaccactggaatgggctggctatgaaggaggtgattaccttgtcacgcagtgaggtatgcaagaatagcaggatgactgccactaggcagagatgttgatgagaagcaaactcaaatattctatagttggttagaataggcagaattgagttcctatccttactctgttttgttttgttttgttttgttttaaatacagatgtggtctcactgtgttgcctaggctggtctcgaactcctaggctcaagcaatcctcccacctcagccttccaaagtgctgggattacagacattaagccaccatgcctggcctcttattctgttctttaagcaggtgtagatgtgtgactgtaagttacttcacctctctgtgtttgtagttgatatgtcagataagtataatagcggtacctatctcatagttatttgccatgattaaatagtgctggtcaagcatttagccaatgctcatctcataataagcatccaataaactatagctgcccattaaagcctcttacagggctaggcacagtggcttacacctgtaatcccaatgctttgggaggctaaggtgggaggattgcttgagaccaggagtttgagattagcctggacaacatagcaagaccttatcactacaaaaaattccaaaaaattagctggacatggcacatgcctgtaatcccagctactcaggagcccaaggcaggaagtttgcttgagcccagaagtttgaggctgtagtgagccatgatcatgccactgtactgcagcctgagcggacagagcaagactctgtctcaggaaaaaaaaaaaaaagtaagtaattaaaataaaaattataaataacaaaaatttaaatcctcttatagtcctaggattgtttttataggctccatcaggccccagggagcataagctttctgctggcaatcctgaatgctcccctcagcccgttgctgactttgtgccccaacccccttcccagggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatgtgagatcctagggcaggggtggggtaaagggcccgggggtgtggggactggccttaagcccaggagagagttcacttatgccccacttccgttctgtgcctttgcagattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcaggtgaggagtaggggggatgggagcccttgggaggggacagtagagcaggaaggactgtcccttgagtgcaagaccaagggggtgttgattcccctgctgctgtggggagtgcctttaacctctgggaattgtccttggccgtctgcagggaagagcagagctgtgctgtctagagttctcaggggttgggtggagggatgtggggtgatgtgaggaaggaaggaggggctctgactgttttacccctccagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccaggcactgggctactactatgtgctggagccacgggtgagggtcaggacacccccaggccctgtctatggtcacacacacccgcagtgctccctctgctttctgataacaaagatcacccaaagtaaccctgttcctacagccacgccagcccagtccaggattccactcagacttatctgtacatgggtatcaggaaaacgagccccagactccagatatgtggccaataggtccctctagcctatcctatttcccgtggtttcttttcctctgactccccatcttctaatgctgcatttggtgagaatcgaatgtgtggcctttgggacaagtccttcacccctcttccatgggctcctctgtcaaattatagtggtggtttcattcagctcctggacagccagtccctcaatggacaggcctcctggagatgactgaagtccctacactccctctctgtgatagggtggtgggttgaaggctgcacaaagaaaaacaagccccagcagcattttgctcccttcgccccaacctgagaatttctacaggctggctgccctctagcgtccatttagggacagtgccatcaaggtttagggcttggtgctgggaccagacagactgcacttcccatcttggctcctctgcttgccatttgtgtggcttttggacaggttctttagcttttctaaacctcaacgttccatccctaaaaacctcaaatagtggtggtgagatttacatttgtgtttataaaacacttagcatgctgcctgattcatactcagtgctctgaaaatgctggctagatgttatatgtgataataattattattgttatgattatcacccacatggtggccggcactcacccatctacatcccctacttcatccaacttttccgacaacattgcttccctgatgggacatgggtctcgtttgatttaccaaccccttttgacttaggaggaaccttcagcattgttctttctcttttcctcaggtggtagatgggaccccctgttccccggacagctcctcggtctgtgtccagggccgatgcatccatgctggctgtgatcgcatcattggctccaagaagaagtttgacaagtgcatggtgtgcggaggggaccgttctggttgcagcaagcagtcaggctccttcaggaaattcaggttctttcaccatcattcctccctctaccttcctggacattctgggtcctagtgatgcagaggggaactcagcactgtttccactctcaaagagctcacagtctagcgggagatagacgagtaggcagctacagtgcggtttcccatgtattcatccgaagttggaacagggtgttgtgagtgcccttgtgggcatcagggaaggcttccttggaggtgacctctgagctgagtctcagaaggagggtaggaattagacaggtaccaaggaagtggggcttggaagggcaactcaataagggggcagtgggtgtaaaggtccagagataagaggcagtagggcacatgtgcatgtagctctgatccagaccgaacagctcacataggattgggtgacagccatctacacacatgtacacacatacagtcacataagcacagctcagaaggctgaggggattcaggtgatcctgagctccccatctactccttccacacaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccggctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataacactcagcatctagtaagtgctcaacaaatagcagctgctgttacttactgttatcaaatttctgtccacatccactctccatatgcacttgaaggtggcaaagatccacaaccatggtgcctgcctttatcctcagggtccgttcctttggttggcagacccctatcctgggttctgagggaccaacagagaaaggaaaattccatccctcacctctggaagttcccaatcacaggaaggaaacatagtaagcacgtggctacaaatacaattgacaagaacatgaaggtgcaggataacaaga

SEQ ID NO: 3 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGTPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQARALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 4 - DNA, 72 bp, Artificial
  probes
gggccacccacattcttgtccggcagcagggaaaccgctggccaccggagcatctacttggccctgaagctg

SEQ ID NO: 5 - DNA, 19 bp, Artificial
  primer
gggccacccacattcttgt

SEQ ID NO: 6 - DNA, 22 bp, Artificial
  primer
catctacttggccctgaagctg

SEQ ID NO: 7 - DNA, 85 bp, Artificial
  probes
ctggcaccatcaatggagatccggagtcgagtggcatctctgcacttgggatgggggagccctgttaggcgtgttacaatatcgg

SEQ ID NO: 8 - DNA, 21 bp, Artificial
  primer
ctggcaccatcaatggagatc

SEQ ID NO: 9 - DNA, 24 bp, Artificial
  primer
ctgttaggcgtgttacaatatcgg

SEQ ID NO: 10 - DNA, 74 bp, Artificial
  probes
cactctccatatgcacttgaaggtggcaaagatccagcaaccatggtgcctgcctttatcctcagggtccgttc

SEQ ID NO: 11 - DNA, 24 bp, Artificial
  primer
cactctccatatgcacttgaaggt

SEQ ID NO: 12 - DNA, 22 bp, Artificial
  primer
cctttatcctcagggtccgttc

SEQ ID NO: 13 - DNA, 78 bp, Artificial
  probes
tcgtgtttccagagaagctcaacggcagcgtcctgcctggctcgggcgacccctgccaggctgttgtgccgcttgcag

SEQ ID NO: 14 - DNA, 23 bp, Artificial
  primer
tcgtgtttccagagaagctcaac

SEQ ID NO: 15 - DNA, 18 bp, Artificial
  primer
ctgcaagcggcacaacag

SEQ ID NO: 16 - DNA, 4342 bp, Homo sapiens
gggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcgcccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggacctgtgtggagtctccacttgcgacacgctgggtatggctgatgtgggcaccgtctgtgacccggctcggagctgtgccattgtggaggatgatgggctccagtcagccttcactgctgctcatgaactgggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatgggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccgggcactgggctactactatgtgctggagccacgggtggtagatgggaccccctgttccccggacagctcctcggtctgtgtccagggccgatgcatccatgctggctgtgatcgcatcattggctccaagaagaagtttgacaagtgcatggtgtgcggaggggacggttctggttgcagcaagcagtcaggctccttcaggaaattcaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccggctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataacactcaaaaaaaaaaaa

SEQ ID NO: 17 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGAPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQARALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 18 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGTPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQARALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNAGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 19 - protein, 837 aa, Homo sapiens [sequence omitted]